(19) United States
(12) Patent Application Publication
(10) Pub. No.: US 2016/0314432 A1
(43) Pub. Date: Oct. 27, 2016

(54) Systems and Methods for Managing Inventory for Health Care Offices

(71) Applicant: Sowingo.com Corp., Toronto (CA)

(72) Inventor: Ravipal Singh Bhatti, Mississauga (CA)

(73) Assignee: Sowingo.com Corp., Toronto (CA)

(21) Appl. No.: 15/139,933

(22) Filed: Apr. 27, 2016

Related U.S. Application Data
(60) Provisional application No. 62/153,358, filed on Apr. 27, 2015.

(51) Int. Cl. G06Q 10/08; G06F 3/0482; G06Q 50/22

(52) U.S. Cl. CPC G06Q 10/087; G06Q 50/22; G06F 3/0482

(57) ABSTRACT

Systems and methods are provided to manage inventory of products used in health care practices and organizations. The systems include tools to track inventory, purchase inventory and to predict how much inventory will be used. The method includes obtaining a schedule of future health care procedures. The method predicts an amount of a product that will be consumed by comparing each procedure against a corresponding procedure profile obtained from a procedure profile database. Each procedure profile includes product usage information of products expected to be consumed in a given procedure. This information is then used to predict when orders for replacement units of the product should be placed, to prevent insufficient supply of the product for a scheduled procedure. A graphical user interface is presented to order the replacement units by a determined date and for a determined quantity. Vendors interact with the system to sell the products.

### CROSS-REFERENCE TO RELATED APPLICATIONS

This application claims priority from U.S. Patent Application No. 62/153,358 filed on Apr. 27, 2015, and titled “Systems and Methods For Managing Inventory For Health Care Offices”, the entire contents of which are hereby incorporated by reference.

### TECHNICAL FIELD

The following invention or inventions generally relates to managing inventory for health care offices.

### DESCRIPTION OF THE RELATED ART

Health care organizations and practices use a variety of different products to help patients. The types of products vary by the field of health care. For example, in the field of dentistry or oral health, typical products include gloves, mouth mirrors, medicine, gauze, needles and dental burrs. For a general practitioner or a family doctor, typical products include tongue suppressors, gloves, syringes, cotton swabs, test tubes, urine sample cups, hand sanitizer, bacterial test kits, alcohol swabs, drugs, and disposable ostocope speculas. Other fields of health care may use other types of products.

Health care organizations and practices need to ensure they have a sufficient amount of products required to perform certain procedures, tests, operations, etc. Product is typically ordered or purchased, and then stored at the location of the organization or practice. When there is an insufficient quantity of product stored in inventory (e.g. the product has run out), or preferably before there is no more product in inventory, then a person from the health care organization or practice will order more product to restock the inventory.

Computing systems are used for keeping track of the inventory and ordering the inventory, but even with current computing systems these are a difficult and time consuming processes.

### BRIEF DESCRIPTION OF THE DRAWINGS

Embodiments of the invention or inventions are described, by way of example only, with reference to the appended drawings wherein:

FIG. 1A is a system diagram showing example components and devices used to manage inventory for health care offices.

FIG. 1B shows some of the example user devices of the system in FIG. 1A, including a handheld scanner.

FIG. 1C shows an example mobile device as per the system in FIG. 1A.

FIG. 2 is a diagram showing example components of a vendor module.

FIG. 3 is a diagram showing example components of an inventory management system (IMS) user module.

FIG. 4 is a diagram showing example components of an administrator module.

FIG. 5 is a diagram showing an example database configuration of the IMS.

FIG. 6 is a flow diagram showing example executable instructions for displaying inventory information for a specific office associated with a user.

FIG. 7 is a flow diagram showing example executable instructions for removing and adding units of inventory.

FIG. 8 is a flow diagram showing example executable instructions for adding in inventory or creating inventory, either using a scanned data or manually inputted data.

FIG. 9 is an example of data relationships between an appointment schedule and inventory.

FIG. 10 is a flow diagram showing example executable instructions for determining when to order inventory based on the currently available inventory and the predicted use of inventory, as per an appointment schedule.

FIG. 11 is a flow diagram showing example executable instructions for computing the date to order product and for computing the amount to order.

FIGS. 12-26 and 28 are example graphical user interfaces (GUIs) that are displayed to a user through a website for managing inventory.

FIG. 27 is a flow diagram showing example executable instructions for automatically updating the inventory database based on tracking numbers and shipment status.

FIGS. 29-36 are example GUIs that are displayed to a user through an application for managing inventory.

FIGS. 37-41 are example GUIs that are displayed to a vendor through a website.

FIG. 42 is an example GUI that is displayed to an administrator of the IMS.

### DETAILED DESCRIPTION

It will be appreciated that for simplicity and clarity of illustration, in some cases, reference numerals may be repeated among the figures to indicate corresponding or analogous elements. In addition, some details or features are set forth to provide a thorough understanding of the embodiments described herein. However, it will be understood by those of ordinary skill in the art that the embodiments described herein are illustrative examples that may be practiced without these details or features. In other instances, well-known methods, procedures and components have not been described in detail so as not to obscure the invention illustrated in the examples described herein. Also, the description is not to be considered as limiting the scope of the example embodiments described herein or illustrated in the drawings.

It is herein recognized that one of the significant obstacles that all practitioners and administrators have in health care is the ability to manage their supplies. The task is often made the responsibility of ancillary staff members rather than the practitioner due to time constraints. The lack of software, amount of supplies required and time constraints of staff make the process of keeping supplies organized extremely difficult, inefficient and cost-prohibitive.

It is herein recognized that various factors make the management of supplies or inventory difficult, including: the variety of supplies, different storage locations, different offices for a single practice, expiry dates of certain products, different usage/consumption rates for different products, different practitioners within a single practice or office, and different vendors for different products. Different staff members may also consume product at different rates. For example one staff member may consume a lot of product for one procedure, while another staff member consumes less product for the same procedure.

It is also herein recognized that there are few computing systems that are able to track the usage or consumption of products, as well as the stocking of products. Typically, these systems require a staff member to manually enter in data into a computer (e.g. via a keyboard), which is time consuming, may be delayed, and may be inaccurate. In other words, current computing software for tracking consumption of products do not provide an accurate and near real-time inventory status.

It also recognized that tracking custom products or items may be difficult, since custom products or items may be few in number or even unique to a given patient. For example, dentures for a patient are unique.

It also herein recognized that calendar systems, or appointment scheduling systems, for a health care practice or organization are typically not linked to inventory management. Tracking future use of products in comparison against available inventory is difficult.

It is also herein recognized that typically the computer processes for purchasing products via the Internet are separate from a company's typical inventory management software. This isolation between the computing systems makes it difficult to reconcile data, and typically requires a staff person to manually input data about shipped or received products into the company's inventory management software.

The proposed systems and methods are for an inventory management system (IMS) for health care.

The IMS in an Internet-based computing system that focuses health care supplies and redefines the health care industry value chain. The IMS also provides a centralized e-commerce solution for purchasing health care supplies. The IMS may also develop a peer-to-peer network for jobs and practice opportunities. The IMS may also include a portal for real-time documentation of regulatory compliance and safety standards. The IMS also provides information and software processes for health care professionals and staff to make empirical and cost-effective decisions using comprehensive and up-to-date information.

The proposed systems and methods are operable, from a user's perspective, on various types of computing devices, including mobile devices (e.g. smart phones, cell phones, tablets, etc.).

The proposed systems and methods include a scanning feature, which scans a product using a camera device on the mobile device, or pairing the mobile device (or other computing device) with a handheld scanner. For example, the scanner and the computing device are in wireless or wired communication with each other. For example, a barcode identifying the product is captured and entered into the system by taking a photograph of the barcode, or by using a scanner. The barcode, for example, is a universal product code (UPC).

In an example embodiment, after a product is entered into the system, the product may by default be visible to other users. In this way, when another user later scans in the same barcode, the same product will be displayed to the other user. Entering new products into the system helps to build the database of UPC codes and save time for other users so that they do not have to enter in the same information again.

The proposed systems and methods also recognizes that a user may operate several offices at different locations. The IMS allows a user to keep track of inventory at more than one location, and to consolidate management of inventory across the different locations.

The proposed systems and methods integrates with the calendar or appointment scheduling applications used by the health care organization or practice. In this way, the future use of supplies or products may be estimated or predicted, and action may be taken in advance to ensure that there is a sufficient amount of supplies or product for future appointments with patients.

For example, the IMS includes a procedure profile, which is a mapping between a given procedure and the associated amount of supplies that is estimated to complete the given procedure. For example, for a hygiene procedure, the associated estimated number of supplies include: 6 gloves, 4 sterilization pouches, and 10 pieces of gauze. The number of supplies associated with each procedure may be customized by the user.

In another example embodiment, the level of supplies required for each “Procedure Profile” will be determined though machine learning and data analysis captured by the IMS and other supply utilization mechanisms.

In another example embodiment, the “Procedure Profile” is directly tied into the “Minimum thresholds” for each supply. When the minimum threshold is reached for a supply, an “automatic” re-order mechanism can be activated to prevent shortages.

In another example embodiment, the “Procedure Profiles” are linked into EHR (electronic health records) software.

In another example embodiment, the proposed systems and methods include a platform for selling and buying supplies or products. Through this marketplace the user will have access to hundreds of suppliers, also called vendors, and thousands of supplies. The marketplace also allows the user to see the same supply from multiple suppliers thus allowing them to do price-comparison shopping.

Another aspect of the IMS includes a portal for vendors to manage the sale of products, promotions, orders, and returns. Another aspect of the IMS includes a portal for an administrator to manage information from vendors and users.

It will be appreciated that the IMS described herein is especially applicable to health care organizations and practices. Non-limiting examples include hospitals, clinics, doctor offices, veterinary clinics, optometry clinics, nursing homes, chiropractic clinics, laboratories, and therapy clinics. In general, organizations or businesses that have appointments and scheduled procedures with patients or clients may benefit from using the systems and methods described herein. While many of the examples described herein relate to oral or dental healthcare, the IMS is applicable to many different types of health care.

In an example embodiment of the IMS, there are different versions. For example, an enterprise edition prevents sharing of information (i.e. UPC Codes) to other users and also contains other features that may or may not be part of a basic edition of the system.

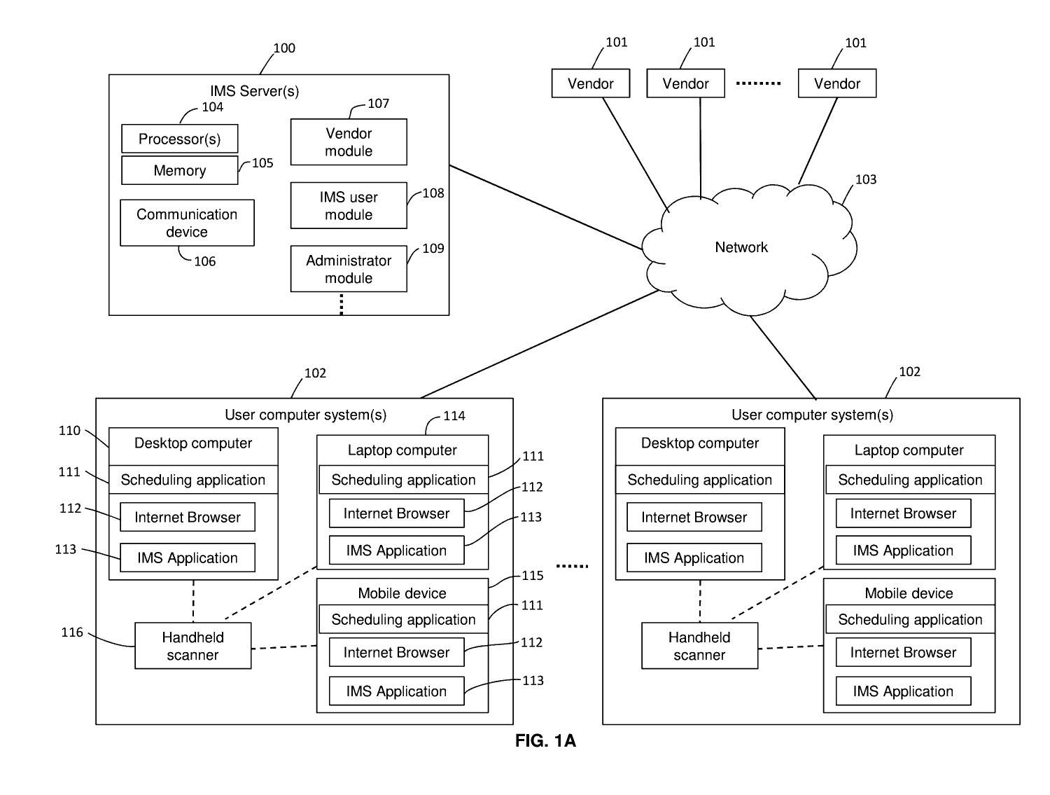

Turning to FIG. 1A, an example embodiment of a system is provided for managing inventory. The system includes one or more servers 100, one or more computing devices 101 used by vendors, and one or more user computing systems 102. These components are in data communication with each other through a network 103. The network, for example, is the Internet.

It is appreciated that multiple servers 100 may operate together to perform the functions of the inventory management system. For example, the servers may perform the same functions as each other, or may perform different functions. An embodiment of a single server and another embodiment involving multiple servers are herein referred to as “a server” and “the server”.

The server 100 includes one or more processors 104, memory 105, and a communication device 106. It will be appreciated that other computing devices described herein also have such components or variants thereof. The server also includes a vendor module 107, an IMS user module 108 and an administrator module 109.

The vendor module 107 includes computer executable instructions and data that is used by a vendor to upload and manage the selling of products to users. The IMS user module 108 includes computer executable instructions and data that is used by a user to manage inventory, purchase new inventory from vendors, and perform other features. The administrator module 109 includes computer executable instructions and data used by an administrator to view and manage the overall inventory management system.

A user uses a computing system 102 to access the server 100. The computing system or systems may include one or more computing devices, such as a desktop computer 110, a laptop computer 114 and a mobile device 115. Non-limiting examples of mobile devices include tablets, notebooks, personal digital assistants (PDAs), wearable computers, smart phones, mobile phones, etc. Preferably, although not necessarily, the mobile device includes a camera device and may further include image recognition software. The computing device or devices include a scheduling application 111 for making and tracking appointments with patients, an Internet browser 112, and an IMS application 113. A given one of these user devices include a processor, a memory, a display screen and a communication device for accessing the Internet.

Through the Internet browser, a user can access the IMS website provided by the server 100. Alternatively, or in addition, a user accesses data from the IMS server 100 using an application that is installed on a given computing or mobile device.

The user computing systems may also include one or more handheld scanners 116 which are in data communication with one or more of the computing devices 110, 114, 115. The scanner is able to take an image of a product or scan a product using light beams. For example, the scanner is a barcode scanner and wirelessly communicates with a mobile device or other computing device over BlueTooth™. The user uses the handheld scanner to conveniently scan a barcode on a product to keep track of the products in inventory, the products being consumed or used, and the products that are being added to the inventory. In an example embodiment, the barcode is of the one-dimensional type. In another example embodiment, the barcode is two-dimensional. In another example embodiment, the scanner is a radio frequency identification (RFID) scanner that is used to read an RFID tag located on a product.

For example, as shown in FIG. 1B, the handheld scanner 116 communicates with one or more of the other user devices 114, 110, 115 over a wireless data connection 120. For example, the wireless data connection is BlueTooth™. In another example, the wireless data connection 120 is WiFi or infrared, or another type of radio connection. It will be appreciated that each of the handheld scanner 116 and one or more of the other user devices 114, 110, 115 include compatible communication hardware (e.g. a BlueTooth radio, a WiFi radio, and infrared transceiver, etc.) to facilitate the data connection 120. The handheld scanner 116 includes a trigger button 122 to activate a scanning action 121. In an example embodiment, the scanning action 121 is to scan a barcode, and the scanned data is sent by the scanner 116 to one of the user devices over the data connection 120. In another example embodiment, the scanning action 121 is to scan an RFID tag or “read” and RFID tag, and the scanned data is sent by the scanner 116 to one of the user devices over the data connection 120. In another example embodiment, the scanning action 121 takes a picture (e.g. a digital image) of the product and sends the picture to one of the user devices over the data connection 120.

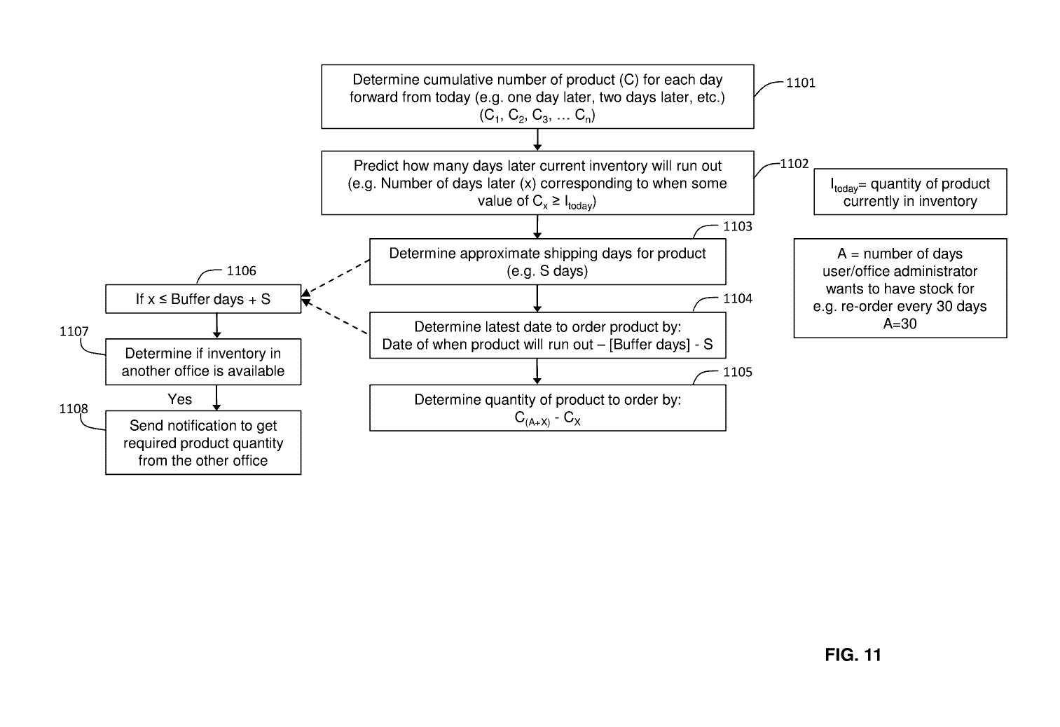

In an alternative example embodiment, FIG. 10 shows a mobile device 115 equipped with a camera or an RFID reader, or both, may perform the scanning action 121 of a barcode, RFID tag, or product, or combinations thereof.

The scanned data is stored in memory of the one more user devices 110, 114, 115 and transmitted to the IMS server 110 over the network 103 via a web portal facilitated by an Internet Browser 112 or an IMS application 113. In this way, the IMS server 100 is able to centrally collect the data from different user devices to help provide a complete and up-to-date inventory.

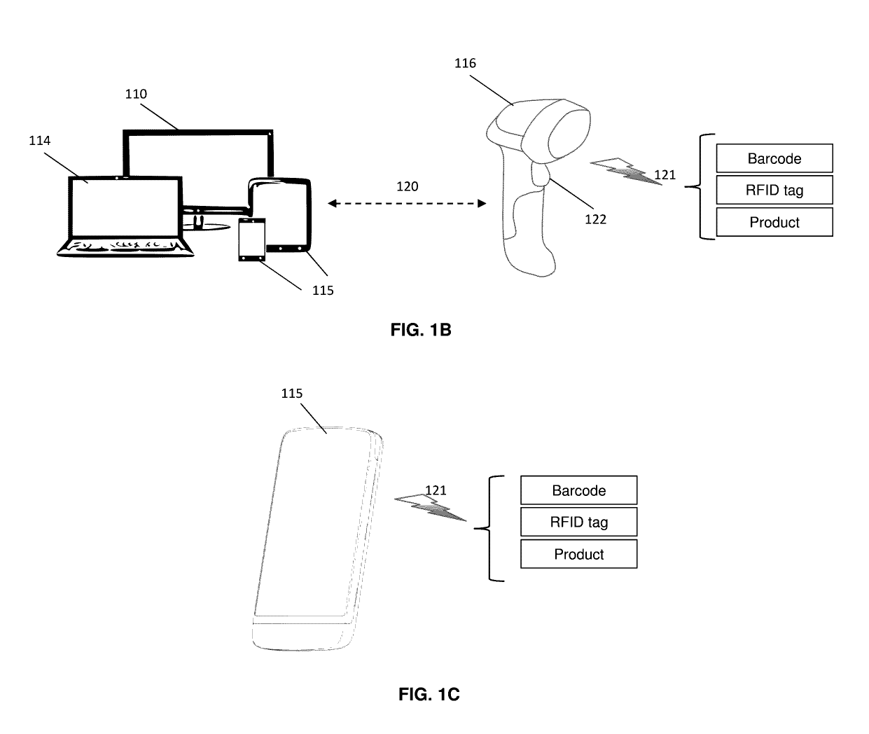

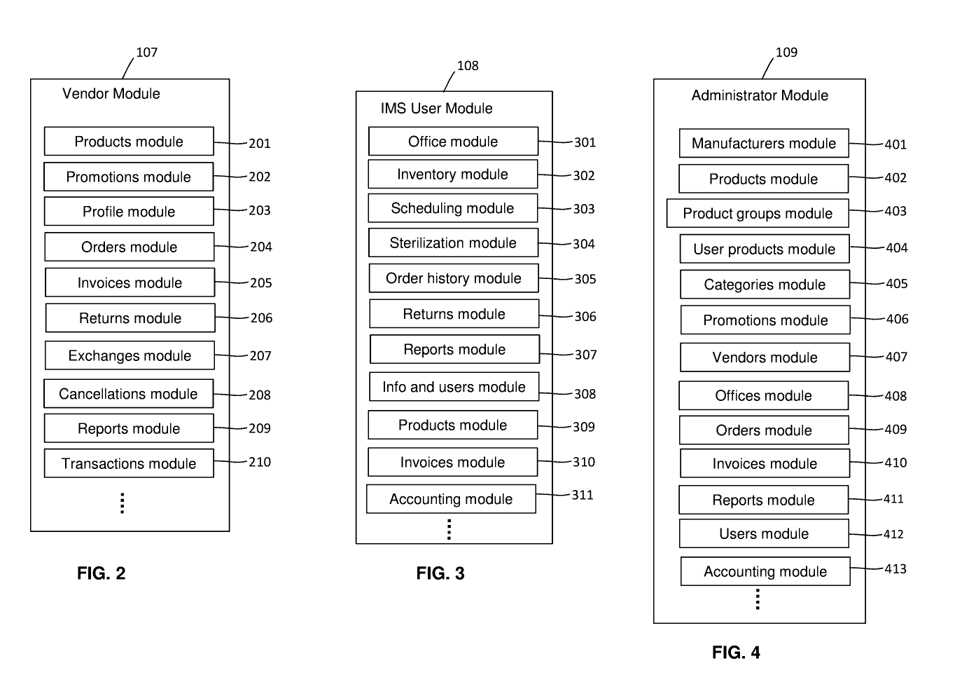

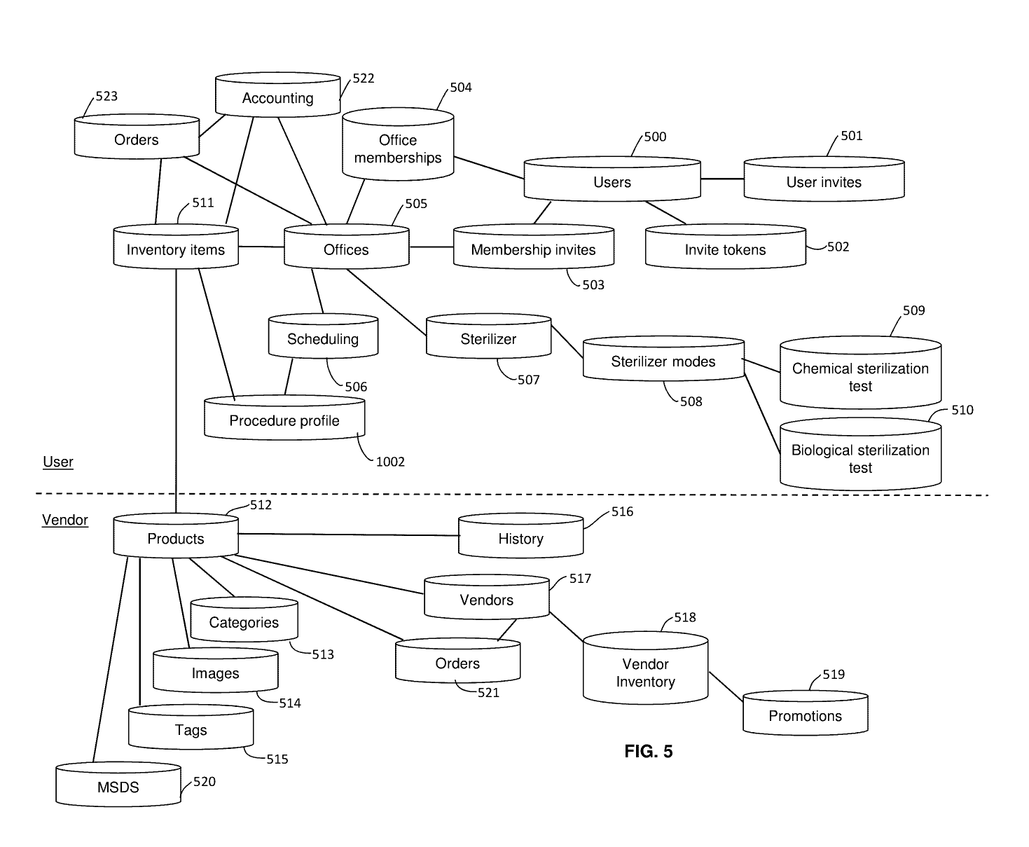

Turning to FIGS. 2, 3 and 4, different modules associated with each of the vendor module 108, the IMS user module 108 and the administrator module 109 are shown.

The vendor module 107, for example, includes: a products module 201 for managing products being sold by the vendor; a promotions module 202 for managing promotions of certain products being sold; a profile module 203 for managing information about the vendor; an orders module 204 for managing orders made by users and shipment of the products; an invoices module 205 for managing the billing associated with the orders made by users; a returns module 206 for managing previously sold products that a user to wishes to return to the vendor; an exchanges module 207 for managing previously sold products that a user wishes to exchange for other products with the vendor; a cancellations module 208 for managing cancellations of orders; a reports module 209 for generating reports regarding the sales of products by the vendor; and a transactions module 210 for managing the exchange of money between a user and the vendor as part of a sale of a product.

The IMS user module 108 includes: an office module 301 for managing different office locations associated with the same user; an inventory module 302 for managing the product inventory associated with the user; a scheduling module 303 for managing usage of products based on the scheduling of appointments and procedures of the user; a sterilization module 304 for managing the testing and upkeep of sterilization equipment; an order history module 305 to manage past orders that were made for products and to track shipments of the products based on the orders; a returns module 306 to manage the products that the user wishes to return to the vendor; a reports module 307 to manage the usage, cost and current quantity of inventory, among other information associated with inventory; an information and users module 308 for managing information about the user; a products module 309 for managing the searching and purchasing process of products; an invoices module 310 for managing the payments and financial refunds of orders 310; and an accounting module 311 to manage the monetary value of products and equipment in inventory for a given user (e.g. amount of monetary value in inventory for a given clinic office location, or a given set of clinic offices).

It will be appreciated that the accounting module 311 includes an application programming interface (API) that is configured to transmit data to a third-party accounting software. In this way, a health care organization is able to use the IMS server 100 to quickly populate data fields in a third-party accounting software.

The administrator module 109 includes modules which are similar to the modules described above. In particular, the administrator module includes: a manufacturers module 401, a products module 402, a product groups module 403, a user products module 404, a categories module 405, a promotions module 406, a vendors module 407, an offices module 408, an orders module 409, an invoices module 410, a reports module 411, a users module 412 and an accounting module 413.

It will be appreciated that the modules may communicate data with each other. For example, the sterilization module 304 communicates with the inventory module 302 to determine which products in inventory require sterilization. In another example, the invoices module 310 and the inventory module 302 exchange data with the accounting module 311 to determine the monetary value of all the inventory within a given office location, or within a given set of offices. In another example, orders module 204, the order history module 305 and the inventory module 302 are used to automatically track shipments of products and to automatically update the inventory database when products have been shipped from the vendor to the designated office location.

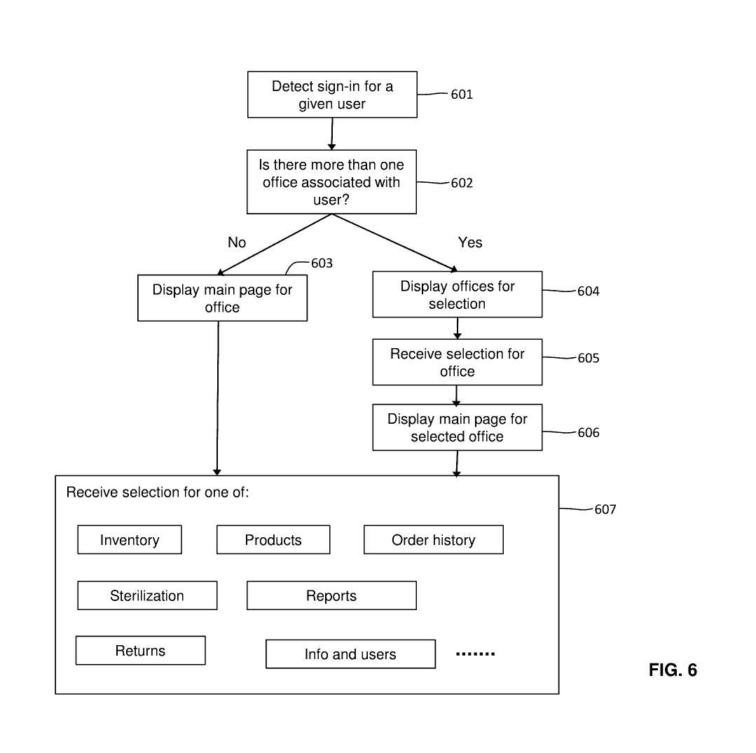

Turning to FIG. 5, an example embodiment of a database configuration is shown for the data stored in memory on the server 100. It includes a users database 500 for storing information about users. Associated with each user are user invitation data 501, invitation tokens 502 used to ensure authenticity, membership invitations 503 and office memberships 504. An offices database 505 includes data about different offices, which are associated with different users and, in particular, the office memberships associated with each user. It is appreciated that a user may be associated with multiple offices. For example, a practitioner has multiple offices at different locations.

Each office is associated with their own inventory data 511, scheduling data 506, and sterilizer data 507.

Associated with each sterilizer are sterilizer modes 508, and these include information about one or more a chemical sterilization test 509 and a biological sterilization test 510.

It will be appreciated that data described in the databases above refer to data that is accessible by a user through their computing system 102. This data, in an example embodiment, is not accessible to a vendor.

The offices database 505 is also associated with an accounting database 522 and an orders database 523. The orders database 523 stores data about orders for products, such as vendor, product, cost associated with an order, order status, a tracking number for the shipment, shipping address, shipping method, etc. The accounting database 522 stores data about the monetary value of inventory in a given office location, or for a given set of offices, or both. The accounting database 522 is also associated with the inventory items database 511 and the orders database 523.

A procedure profiles database 1002 is also included in the memory. Each procedure profile in the procedure profile database includes product usage information of products expected to be consumed in a given procedure. The database 1002 is associated with the schedule 506 of an office and the inventory items 511.

In an example operation of using the procedure profiles database 1002, the server: obtains a schedule of future health care procedures, the schedule identifying a date for each procedure; computes an amount of a product that will be consumed by comparing each procedure against a corresponding procedure profile obtained from the procedure profile database, the product usage information of at least one or more of procedure profiles including a product identifier of the product and a corresponding quantity of the product; computes how many days later from a current date that a number of currently available units of the product in an inventory of the health care office will run out based on the computed amount of the product that will be consumed in the future; determines a date of when to order replacement units of the product and a quantity of replacement units of the product based on at least the predicted number of days later from the current date that the currently available units of the product in the inventory will run out; and provides a graphical user interface to order the replacement units by the determined date and for the determined quantity.

Continuing with FIG. 5, the inventory items associated with an office are linked to a product database 512, which is populated mainly by vendors and the products they sell. The products database 512 is linked to a categories database 513 identifying different categories and sub categories of products. The database 512 is also linked to an images database 514 including images of the products, a tags database 515 for including associated metadata of the products, and an MSDS database 520 that stores material safety data sheets (MSDS) associated with products.

A history database 516 is also linked to the products database showing the past sales associated with products. A vendors database 517 is also linked to the products database 512. The vendors database includes identification of the vendors. The vendor database 517 is also associated with an orders database 521 that stores order information associated with each vendor. For example, the order information includes the selling price of a product, the shipping address, a tracking number associated with a shipment, an order status, and other information associated with an order.

A vendor inventory database 518 includes data about the inventory of products of which they can sell. The database 518 is linked to the vendor database 517. A promotions database 519 is linked to the vendor inventory database 518, the database 519 includes special or featured sale information for specific products.

It will be appreciated that other database configurations and other database associations may be used, including other types of data compared to what is described above.

It will also be appreciated that any module or component exemplified herein that executes instructions or operations may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data, except transitory propagating signals per se. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the server 100, the user computing system(s) 102, the vendor computing devices 100, or accessible or connectable thereto. Any application or module herein described may be implemented using computer readable/executable instructions or operations that may be stored or otherwise held by such computer readable media.

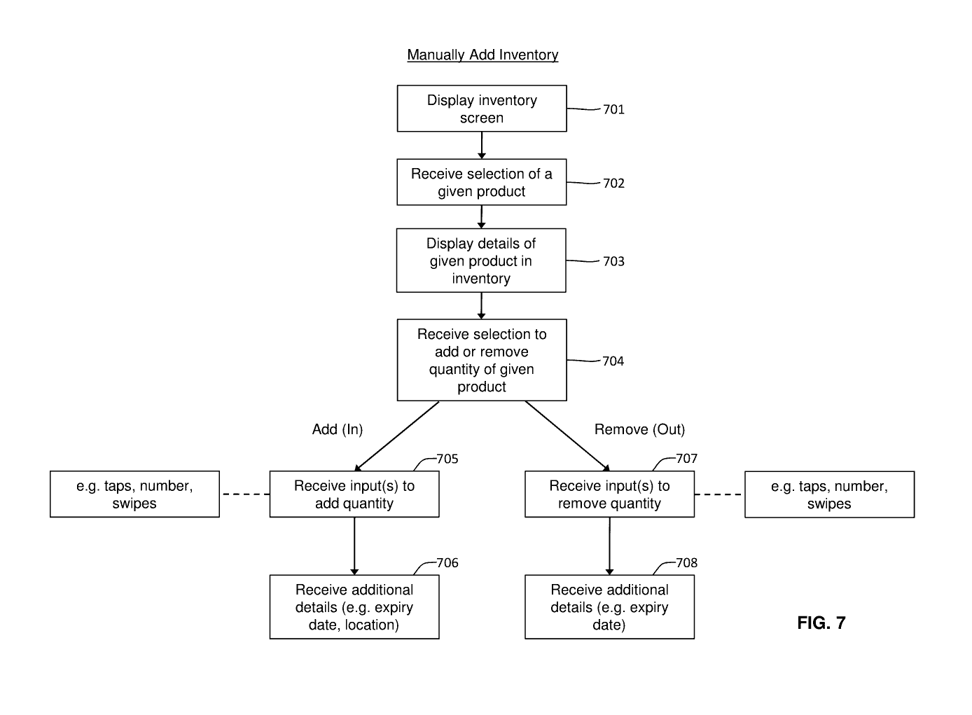

Turning to FIG. 6, example executable instructions are provided for entering a user website for the IMS. The instructions are performed by the server 100. The server interacts with a user's computing device and causes certain actions or invokes certain actions to be performed at the user's computing device, such as displaying information at the user's computing device. At block 601, the server detects a user has signed-in. The server determines if there is more than one office associated with the user (block 602). If not, the main page is displayed for the one office (block 603). If there are multiple offices, the server displays the offices for selection (block 604). The server receives the selection for a given office (block 605) and then displays the main page of the selected office (block 606).

At the main page of the website, a user may select any one of the graphical user interface (GUI) controls related to inventory, products, order history, sterilization, reports, returns, and information about themselves as a user (block 607). Other controls for initiating other operations may be included.

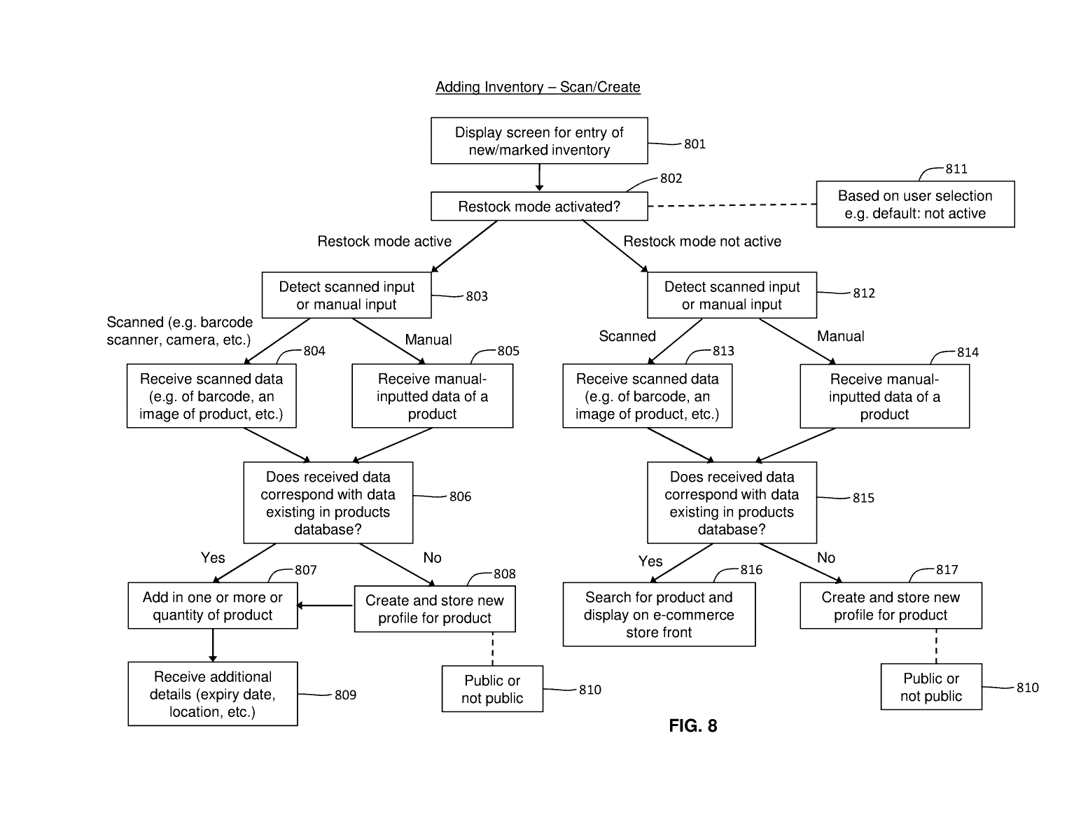

Turning to FIG. 7, example executable instructions are provided for manually adding the records of products into the IMS. These instructions may be executed by an application 113, such as on a mobile device. Alternatively, these instructions are performed by the server 101, via the Internet browser.

At block 701, the IMS application displays an inventory screen. The application receives a selection for a given product (block 702). For example, the user selects a given product. Details of the selected product, which is inventory or was inventory, are displayed (block 703). These details include, for example, the name of the product, the manufacturer, any tags, the location of the product within the office, the current quantity of the product in stock, and the expiry date of the product. At block 704, the application receives a selection input to add or remove quantity of the given product.

If product is to be added (e.g. inventory in), then receive input from user indicating the quantity of product being added (block 705). The application may also receive additional details (block 706), such as the expiry date of the product and the location of where the added product is being stored. In this way, other people may more easily find the product in the office.

If product is to be removed from inventory (e.g. inventory out), then the application later receives input from user indicating the quantity of product being removed (block 707). The application may also receive additional details (block 708), such as the expiry date of the product being removed. In this way, other people may know that there is currently less product available in the office.

The inputs at block 705 and 707 may be in various forms. For example, a user can enter in number using the GUI, tap a number using the GUI, tap a touch screen several times, or make a number of swipes to correspond with each unit of product being added or removed.

The application 113 sends the updated data regarding the addition or the removal of product to the server 100. In turn, the server 100 updates the inventory database in its memory, regarding the addition or the removal of the product from a given office's location. In this way, other people from the same office, or other people that are part of the same practice but from different offices, may be aware almost right away about the current state of the inventory.

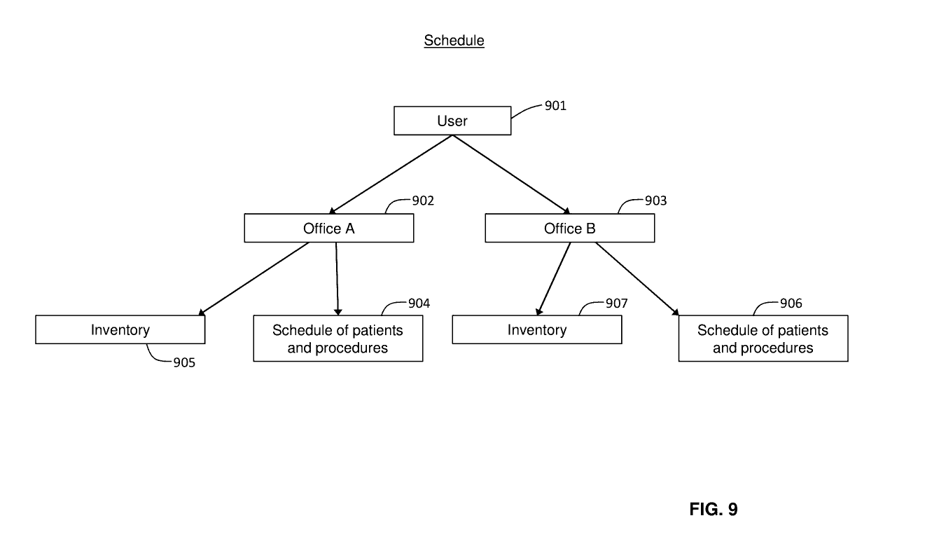

Turning to FIG. 8, example executable instructions are provided for adding inventory by scanning barcodes (e.g. 1 D, 2D, 3D barcodes), reading RFID tags, or scanning a product using a camera, as well as for adding in inventory that has previously not been added to the products database. These instructions are performed, for example, by the application 113. For example, the scanning action 121 in FIG. 1B or FIG. 10 is used to trigger the addition or tracking of a product in an office's inventory database.

At block 801, the application displays a screen for entry of new or marked inventory. The application determines if the restock mode is activated, or not (block 802). In an example embodiment, the restock mode is controlled based on a user selection (block 811). In an example embodiment, the default setting is that the restock mode is inactive, although the user can provide an input to activate the restock mode.

It is appreciated that, in restock mode, existing products are being added back into inventory for the user. In the normal mode, or when the restock mode is not active, entry of product information into the application triggers a search for the product in the product database.

If the restock mode is active, at block 803, the application detects receipt of scanned input or manual input. Scanned input refers to data that has been scanned using a camera device on the computing device or mobile device, or data that has been scanned using a handheld barcode scanner. It is appreciated that known and future known image recognition technologies and handheld scanning technologies are applicable to the systems and the methods described herein. The scanned data identifies a product. For example, the scanned data is UPC code or another visual identifier. In another example, the scanner is an radio frequency identifier (RFID) scanner and there is an RFID tag located on the product.

If the data is scanned, then the application receives the scanned data (block 804). If the data is manually entered, the application receives the manually entered data (block 805).

The application or the server determines if the received data corresponds with data existing in the products database (block 806). This determination is made by accessing the products database 512 on the server memory and comparing the received data (e.g. the scanned data) with the data in the products database. If there is a match, the application adds in one or more units of the product to the inventory database (block 807). In particular, the server accesses the inventory database 511 in memory and executes a write operation to increment the quantity of a given product. If there is no match, the application creates and stores a new profile for the product in the inventory database 511 (block 808), before proceeding to the operation of block 807. It will be appreciated that a user may specify whether or not the newly created product entry in the products database may be viewed by other user (e.g. of different health care organizations or practices), or is intended to be private and only viewable by others under the same user account (block 810).

If the newly added product may be viewed by others, the server adjusts a data tag associated with the newly added product indicating it is available for others to view. The server may further access the products database 512 to populate it with the information about the newly added product, so that a vendor may access the same information and determine if they have it for sale.

After performing block 807, additional details about the new inventory or added inventory is received, such as the expiry date and the location of the product within the office (block 809). A minimum number of product may also be stored in association with the product. For example, the office may require a minimum number of twenty instances of the product to be available in inventory at any given time.

Returning back to block 802, if the restock mode is not active, then the application later detects a scanned input or manual input 812. For example, at block 813, the application receives scanned data. At block 814, the application receives manually inputted data. At block 815, the application or the server determines if the received data corresponds with data existing in the products database. If so, the server searches for the product and displays the identified product on an e-commerce store front for potential purchase by the user (block 816). Otherwise, the server creates and stores a new profile of the product in the products database (block 817). As noted above, the new profile of the product may be made publicly available for other users to view, or is kept private.

For example, if the new product is a unique product or a secret to the user, then the user may wish to keep the product private on the database.

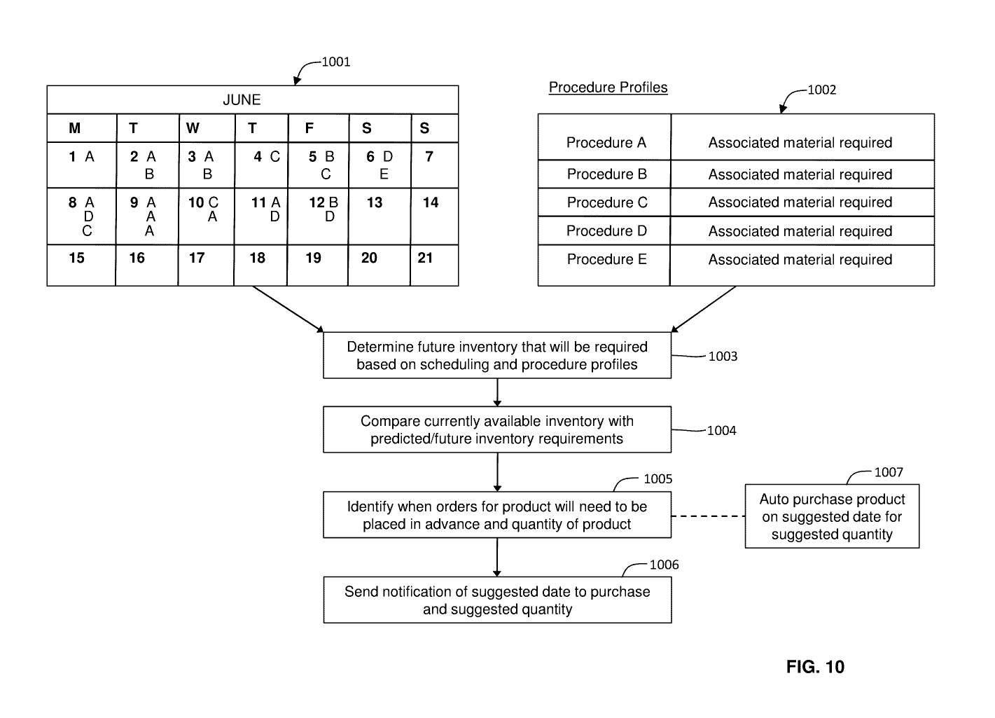

Turning to FIG. 9, an example relationship of data is shown for integrating scheduling and inventory. A user 901 (e.g. a health care practitioner or administrator) is responsible for two offices 901, 902. Office A (902) is associated with certain collection of inventory 905 and a certain schedule of patients and procedures 904. For example, the patients will go to the Office A to have certain procedures performed.

Similarly, Office B 905 is associated with another set of inventory 97 and with the schedule of patient and procedures 906.

Turning to FIG. 10, a calendar or a schedule of appointments 1001 is provided. It identifies the dates of certain procedures, operations, tests, etc., which are herein referred to as procedures. A database of profile procedures 1002 is also considered. Each procedure is associated with a procedure profile. A procedure profile is an association between a given procedure and certain material or supplies required for the procedure. It is appreciated that a user may enter in or input the types of supplies and quantity of supplies associated with each procedure. In another example embodiment, the server tracks usage over time to automatically determine or dynamically adjust the supplies associated with a procedure. In another example embodiment, default settings include an initial number and type of supplies associated with a given procedure, which may be provided by the administrator based on industry practice. Later, the user may modify or adjust the initial number and type of supplies to better suit his or her own health care practice.

The calendar or schedule 1001 and the database of procedure profiles 1002 are inputs into a scheduling-inventory process. At block 1003, the server uses this information to determine inventory that will be required in the future based on scheduling and the procedure profiles. At block 1004, the server compares the currently available inventory with the predicted or future inventory requirements. The server then determines when orders for product will need to be placed in advance, as well as the quantity of product to be ordered (block 1005). Based on this information, the server transmits a notification to the user device 110, 114, 115, which includes the suggested date to purchase a suggested quantity of product (block 1006). Alternatively, if permission and authorization is provided, the server automatically purchases the product on the suggested date and for the suggested quantity (block 1007).

Using the operations of FIG. 10, a user is able to leverage the data from the scheduling system and the inventory system to ensure there will be sufficient amount of product in advance of the scheduled procedures. Therefore, a health care practitioner will not run out of supplies for a scheduled procedure.

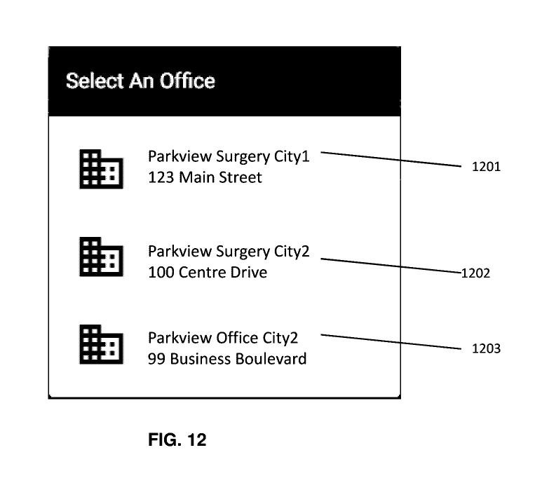

FIG. 11 describes additional details for implementing the operations of FIG. 10. At block 1101, the server determines the cumulative number of a given product being used for each day forward from today. For example, one day later there will be 5 units of a given product being used; two days later there will be 7 units of the given product being consumed; three days later there will be 18 units of the given product being consumed; four days later there will be 24 units of the given product being consumed; five days later there will be 32 units of the given product being consumed; and so forth. This information is based on the schedule and the procedure profiles.

The number of product being cumulatively used is represented by C. The value of C depends on the day forward from today, or some other given day. A subscript of the value C represents the number of days later or forward from today. Therefore, using the above example, C₁=5; C₂=7; C₃=18; C₄=24; C₅=32; and so forth.

At block 1102, the server predicts the number of days later that the current inventory will run out based on the predicted usage. For example, the number of days later is represented by x. The value of x is determined by identifying the smallest value of x for which the following condition becomes true: Cₓ≧I_{today}. The value I_{today} is the quantity of product currently in inventory (e.g. today).

For example, if there are 20 units of products currently available (e.g. I_{today}=20), then according to the above example, C₄≧20. Therefore, the smallest value of x is 4. In other words, it is predicted that the currently available inventory of the given product will be used up or run out in about 4 days from today.

At block 1103, the server determines the approximate number of days to ship the given product (e.g. number of days between date of making the order and date of when the product arrives at the office). This information is obtained from the product marketplace is supplied, for example, by the vendor. The value of the shipping days is represented by S.

At block 1104, the server determines the latest date by which to place the order for the given product. This date may be determined, for example, by computing: (the date of when the product will run out)—(buffer days)—S.

It will be appreciated that the date of when the product will run out is determined by adding the value of x to today's date. For example, if today is June 2 and x=4, then the predicted date of when the given product will run out is June 6.

The buffer days is to account for potential shipping delays as well as the time required to restock supplies in advance of a procedure. For example, the buffer day value may be 1 day, 2 days, 3 days, etc.

For example, the inputs are: today's date is June 2; the date of when the product will run out is June 6; S=1; and the buffer day value=2. Therefore, the latest date to place the order for the given product=June 6−2−1=June 3.

At block 1105, the server also determines the number of units of the given product that should be ordered. The server considers the number of days, represented by A, between which a user or office administrator desires to have to re-order or re-stock inventory. For example, a user may wish to restock every 30 days (e.g. A=30), or every two months (e.g. A=60).

The number of units of the given product that should be ordered is computed by: C_{(A+x)}−Cₓ. In other words, if A=30 and x=4, the server computes the predicted cumulative number of units of product that will be used 34 days from today, and subtracts from this number the cumulative number of units of product that will be used 4 days from today.

In an example embodiment, a buffer quantity may be added to the above computation to account for unpredicted extra usage of product.

In another example embodiment, if there is not enough time for a vendor to ship the required product in advance of a scheduled appointment, then the server may determine if the same product is available at another office location that is associated with the same user (e.g. the same health care practice or organization).

Turning to block 1106, the server determines if the following condition is true: x≦buffer days+S. In other words, even if an order was placed today, the shipped product may not arrive in time for a scheduled procedure that requires the product.

For example, if the buffer days=3, x=4, and S=1, the following condition becomes true: 4≦3+1.

Following block 1106, if the condition is true, then the server determines if inventory in another office is available to take (block 1107). If so, then the server sends a notification to the user to get the required product from the other office in the meantime.

Other aspects of the IMS are described below with respect to the GUIs.

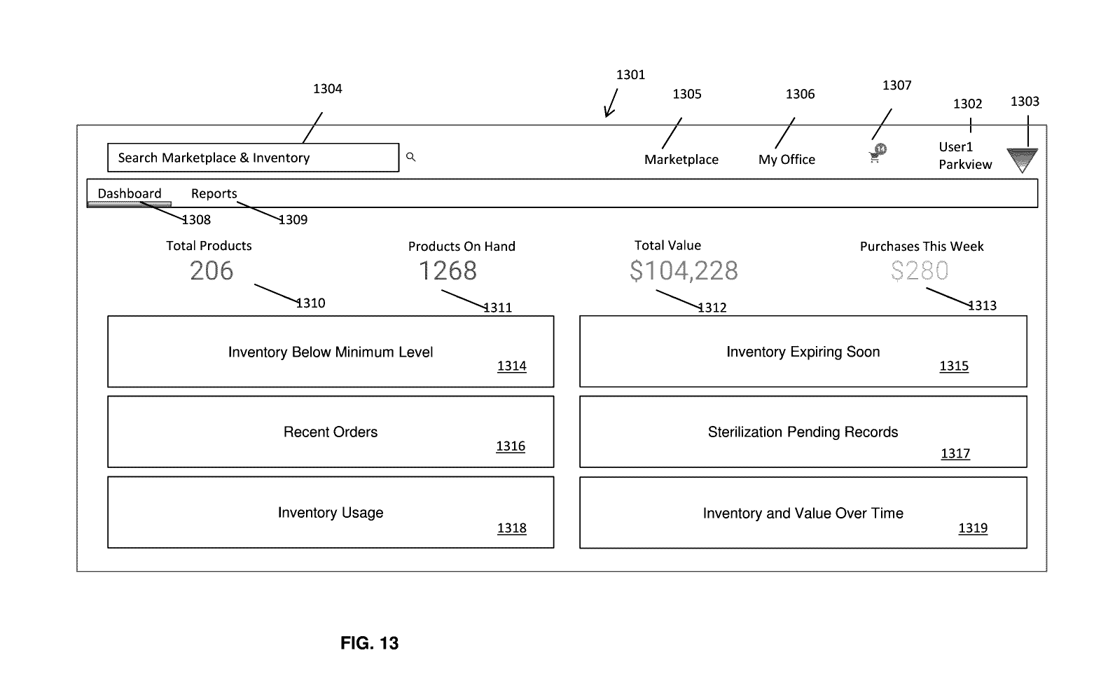

FIGS. 12-26 and 28 show GUIs provided by the server 100 to a user device 110, 114, 115, for example, via an Internet browser residing on the user device. This same content may also be displayed on an application.

Upon a user signing into a user account, the GUI in FIG. 12 is displayed when there are multiple offices associated with a user. The GUI includes selection controls 1201, 1202, 1203 for allowing a user to select one of the offices. It will be appreciated that the offices are physical locations (e.g. a building) for a healthcare organization.

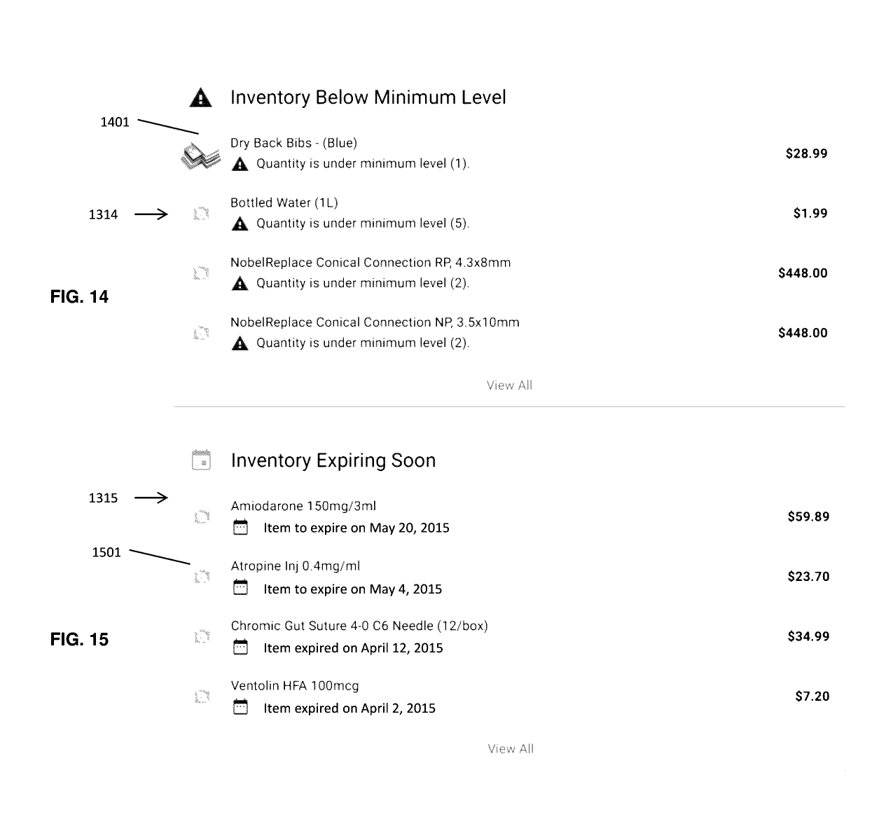

Upon selecting one of the controls 1201, 1202, 1203 to select an office, a dashboard GUI 1301 is shown for the selected office, as per FIG. 13.

In FIG. 13, the user and the office location of the user is shown by the data 1302. In other words, the inventory data, orders, reports, etc. may be navigated and accessed for a specific office for the user. Selecting the control 1303 allows a user to change office locations and to display the data related to a different office location (for the same user).

A search bar 1304 receives input to look for products to purchase on the e-commerce marketplace, or searches for products in inventory for the office, or both. A control 1305 named “Marketplace” may be selected to launch a listing of products or grouping of products, which may be browsed for purchasing. The control 1306 named “My Office”, launches controls to view and modify the data associated with the office (e.g. address, user name, etc.). The control 1307, when selected, invokes the display of items that the user desires to purchase and has put into their virtual “shopping cart”. It also includes a number indicating the number of items currently in their virtual shopping cart.

Controls 1308 and 1309 are for respectively displaying the dashboard GUI 1301 (e.g. currently shown in FIG. 13) and the reports GUI. In other words, selecting control 1309 will display a reports GUI to generate different reports.

The GUI 1301 also displays the total number of types products 1310 at the given office, the total number of products (e.g. instances of products, including of the same type) 1311 at the given office, the total monetary value 1312 of the product in inventory at the given office, and the total monetary value of purchases made this week at the given office for products.

The dashboard includes display sections 1314-1319 showing different types of information, including information related to: inventory below a minimum level 1314; inventory expiring soon 1315; recent orders 1316; sterilization pending records 1317; inventory usage 1318; and inventory and value over time 1319. The details of each of these display sections are shown in the following figures.

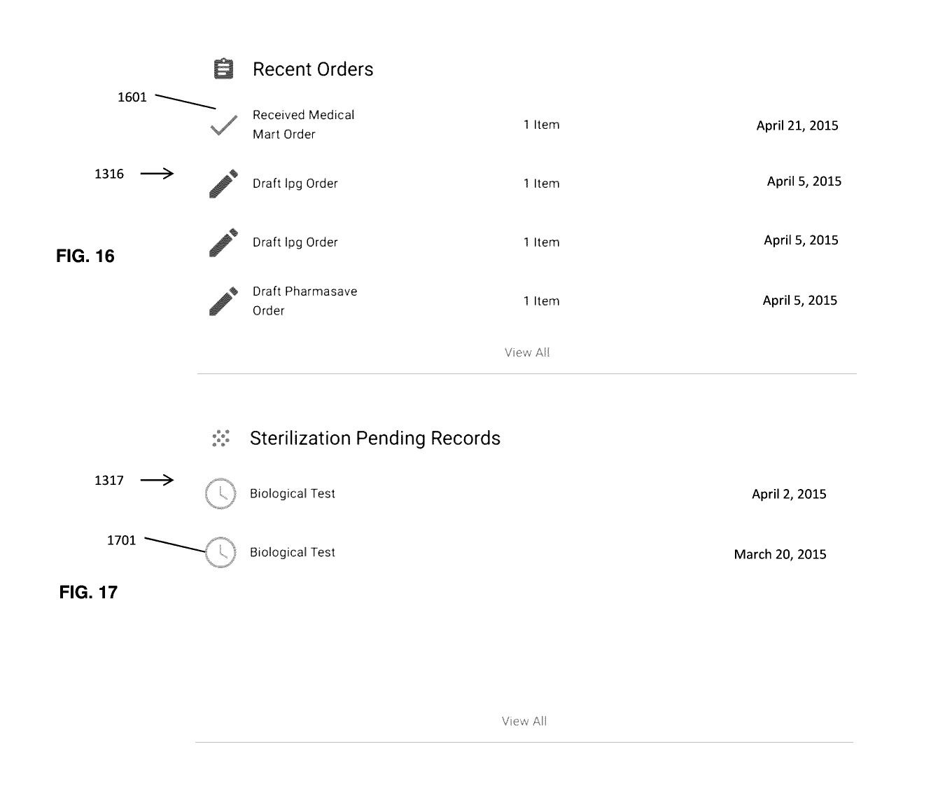

FIG. 14 shows the display section 1314, which includes a listing of entries. Each entry identifies a product that has a number below a minimum level. For example, entry 1401 shows that the quantity of bibs (e.g. a product) is below a minimum level. The entry 1401 includes, for example, a picture of the product, a text description of the product, a symbol indicating a warning symbol, a message indicating the minimum quantity level, and a price associated with re-ordering the product. The entry 1401 is selectable, and upon selection, the server initiates the display of a GUI to re-order the product. There may also be an indication that conveys a given product is expected to be below the minimum quantity level based on expected usage of the product, as determined by the server interacting with the scheduling of procedures and tasks at a given health care office (e.g. as determined using the operations in FIGS. 10 and 11).

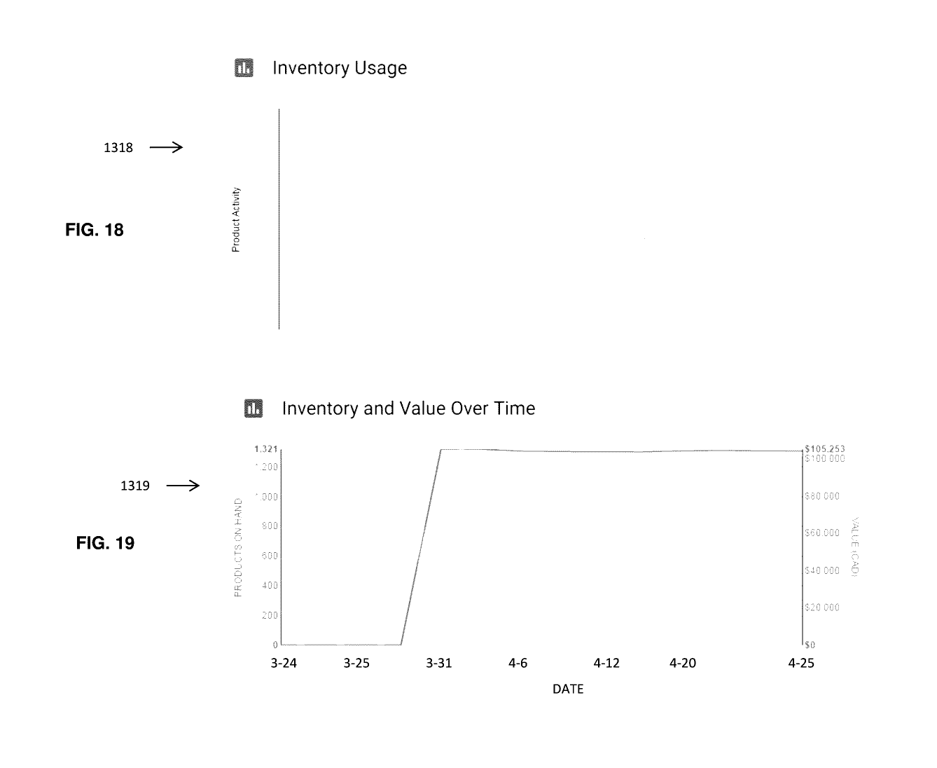

FIG. 15 shows the display section 1315, which includes a listing of entries, and each entry identifies a product that will expire soon in inventory, or has already expired, or both. As will be appreciated, some products have expiry dates. For example, entry 1501 includes a graphic of the product, a text description of the product, a text description of the expiry date (e.g. a past date or an upcoming future date), and a price associated with re-ordering the product. The entry 1501 is selectable, and upon selection, the server initiates the display of a GUI to re-order the product.

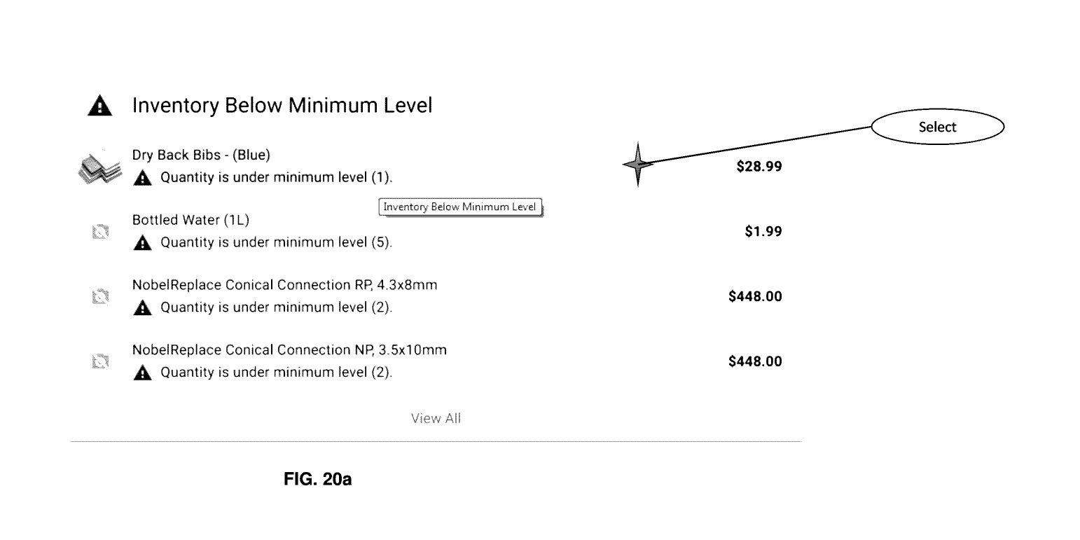

FIG. 16 shows the display section 1316, which includes a listing of entries that each identify recent orders. This is obtained by the server accessing the orders database 523. For example, the entry 1601 includes a symbol identifying the status of the order, a text description of the order and the vendor, the number of items in the order, and the date of the most recent status change. The entry 1601 is selectable to initiate displaying of additional information about the order.

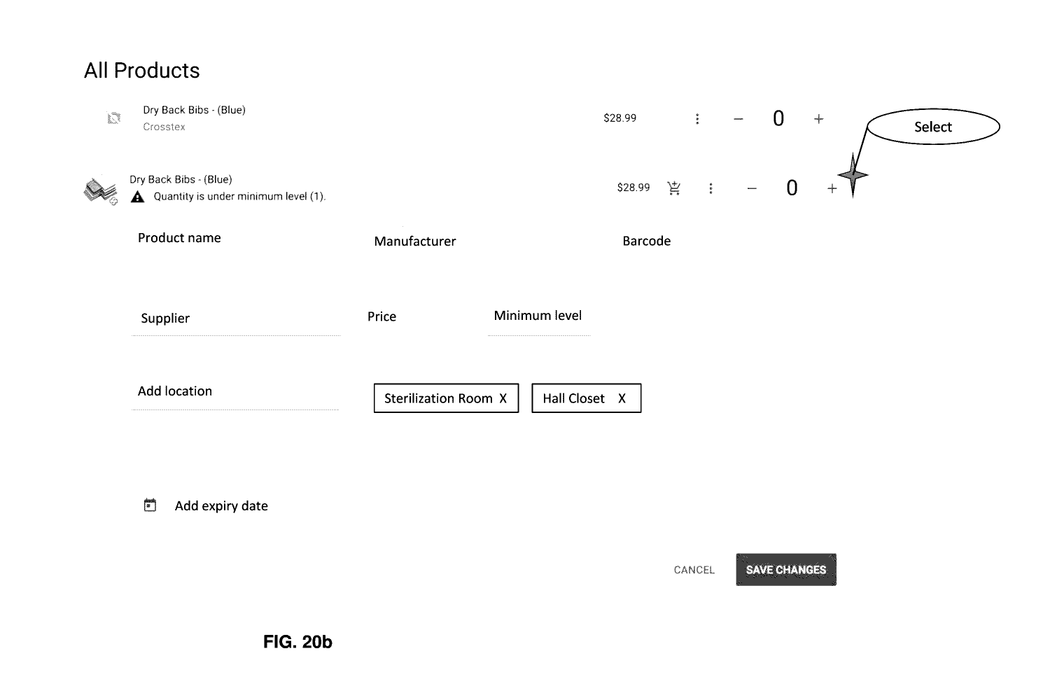

FIG. 17 shows the display section 1317, which includes a listing of entries that each identify pending records of sterilization procedures. This is obtained by the server accessing the sterilizer databases 507, 508, 509, 510. For example, the entry 1701 includes a symbol identifying the pending status of the sterilizer test, a text description of the test, and the date that the test took place. The entry 1701 is selectable to initiate displaying of additional information about the pending test.

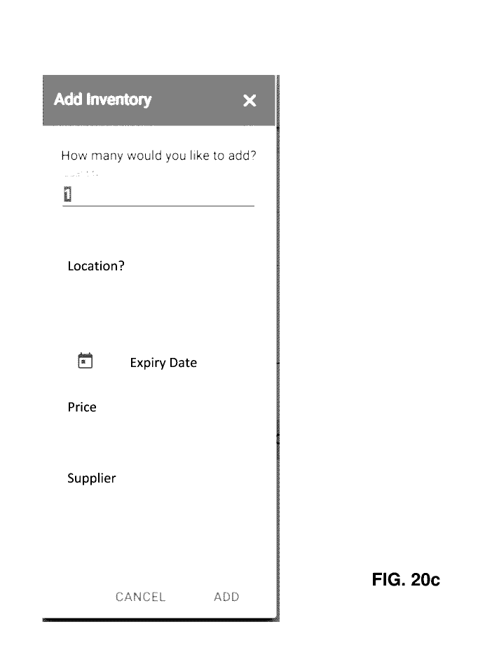

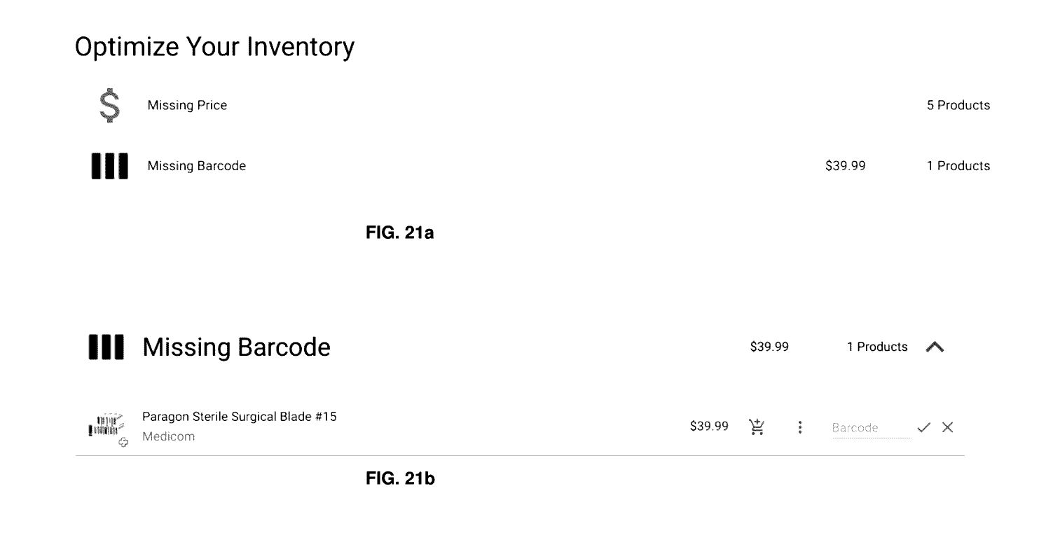

FIG. 18 shows the display section 1318, which shows a graph of the inventory usage over a number of recent days. FIG. 19 shows the display section 1319, which shows a graph of the inventory by number of items and the monetary value of the inventory over time.

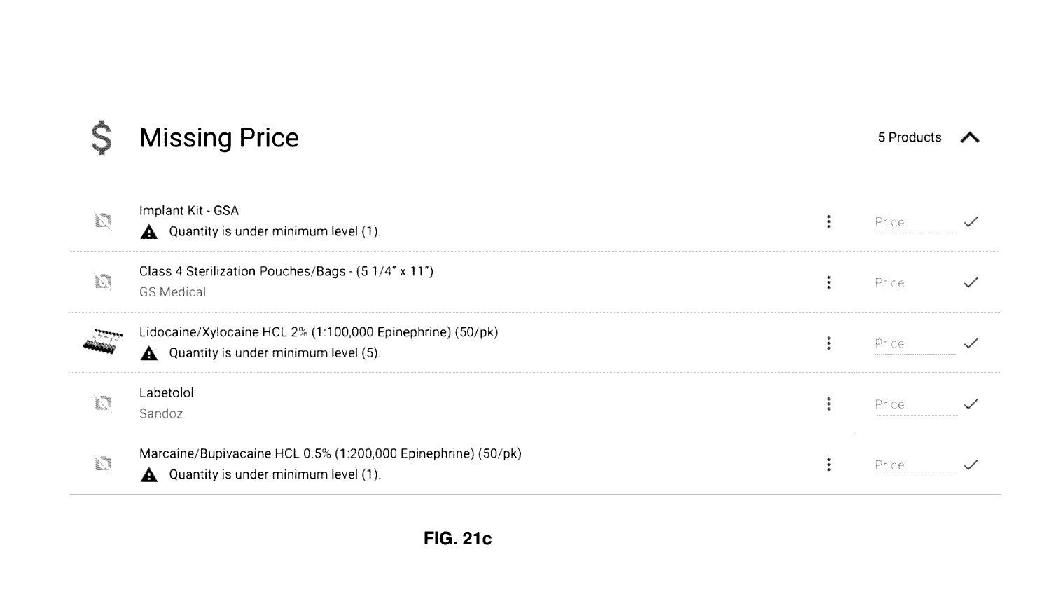

FIGS. 20a-20c show a series of GUI based on user selections of an entry item. For example, in FIG. 20a, selecting an entry item of a product that is below a minimum level initiates display of the GUI in FIG. 20b.

FIG. 20b show details about the deleted product, including the product name, the manufacturer, the barcode, the supplier, the price, the minimum quantity level associated with the product for the given office location, and one or more specific storage location with the given office (e.g. sterilization room, hall closet, etc.). The GUI also includes an option to add an expiry date associated with the product. The GUI further includes controls to add or reduce the quantity of product to inventory. Adding product initiates purchasing the product, or at least adding the product to the virtual shopping cart for checkout using e-commerce. For example, selection of the addition control to add product to inventory triggers the display of the GUI in FIG. 20c.

FIG. 20c shows a GUI for adding inventory. The GUI includes options to determine the number of products to be added, the location within the office for storing the product, the expiry date, the price, and the supplier. Some of these fields may be automatically filled in already based on the information from the GUI in FIG. 20b.

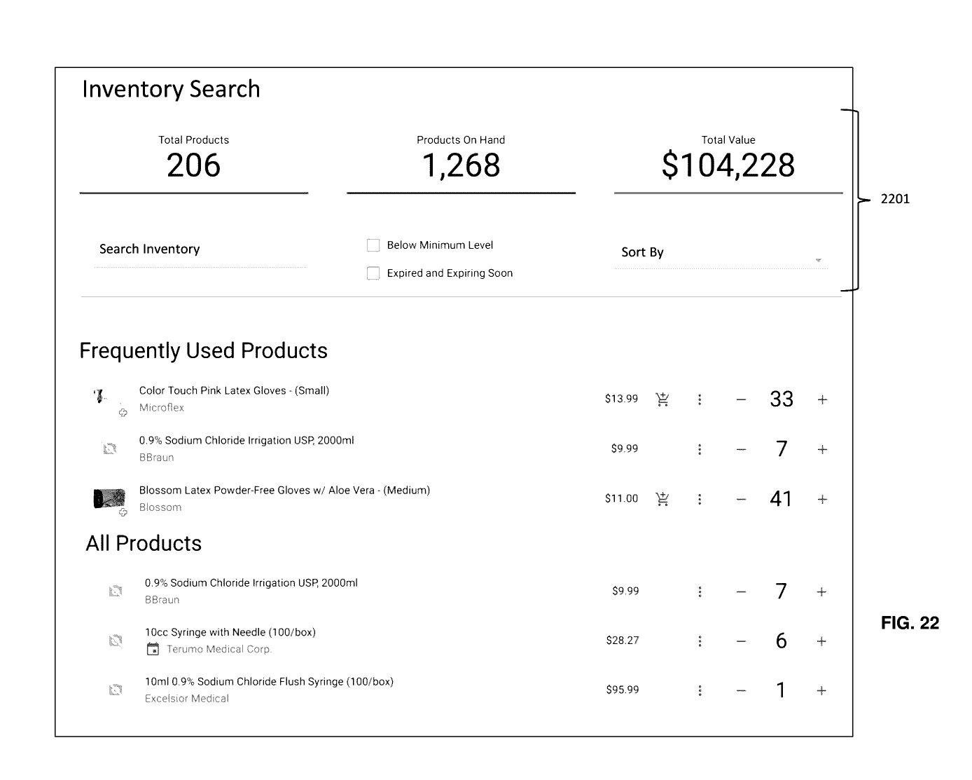

FIG. 21a shows a GUI for optimizing inventory at the given office. It includes a first control for optimizing inventory for products with missing prices, and has associated therewith the number of products having missing prices. It also includes a second control for optimizing inventory for products with missing barcodes, and has associated therewith the number of products with missing barcodes. In other words, the server identifies product entries in the inventory database associated with a given office that have missing information (e.g. missing price or missing barcode), and displays these product entries in the GUI for the user to rectify.

Selecting the second control initiates display of the GUI in FIG. 21b. In FIG. 21b, it shows an entry of the product having the missing barcode. The entry includes an image, a text description, a supplier, a price, an option to add the product to the virtual shopping cart, and an option to add a barcode to be associated with the product. The entry is selectable to trigger the display of additional information about the product. There may or may not be alerts associated with the product (e.g. about to expire, expired, predicted to be soon below the minimum level, currently below the minimum level, etc.).

Selecting the first control in FIG. 21a initiates display of the GUI in FIG. 21c. In FIG. 21c, the GUI includes entries of different products with missing prices. Each entry includes, for example, an image, a text description, the supplier, and an option to add a price of the product. There may or may not be alerts associated with the product (e.g. about to expire, expired, predicted to be soon below the minimum level, currently below the minimum level, etc.).

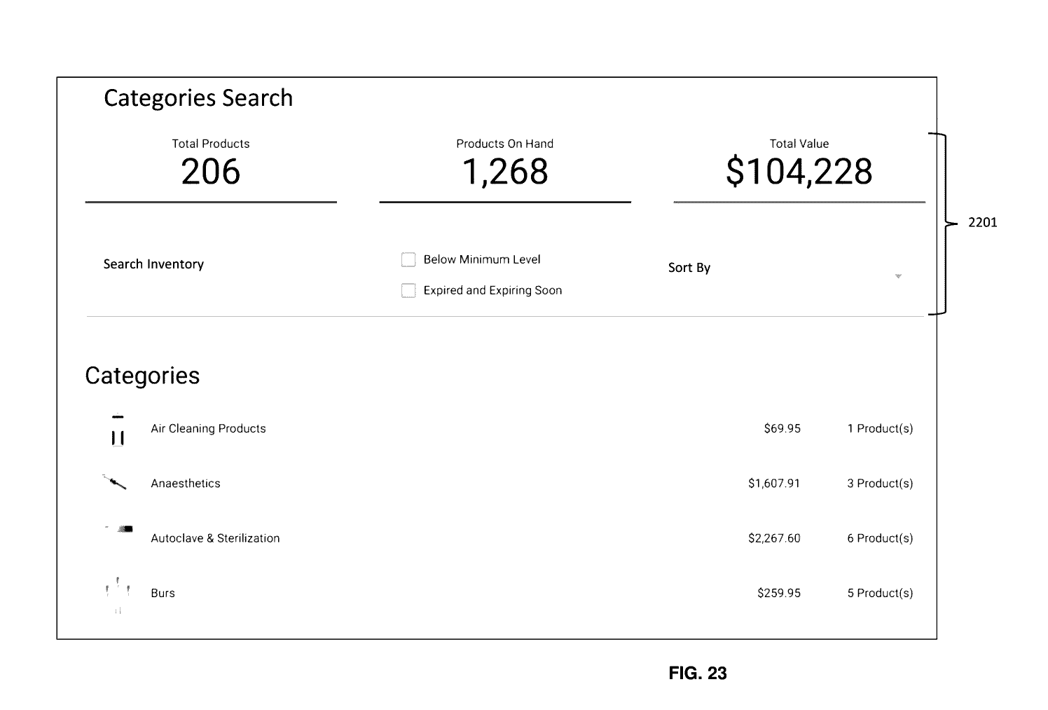

FIG. 22 shows an inventory search GUI. It includes a section 2201 showing the total number of types of products, the total quantity of products, and the total monetary value. These total values are obtained by the server accessing the inventory database and accounting database, and determining the records associated with the given office. The section 2201 also includes a search field to receive input for a query term. The section 2201 also includes a filter for showing items in inventory that are below a minimum level and another filter for showing items in inventory that are expired or are expiring soon. There is also a drop down sorting filter used to sort the results by various parameters.

In addition to section 2201, the GUI in FIG. 22 includes a listing of frequently used products and a listing of all products. Each of the entries are selectable to initiate a detailed display and ordering of additional product using the integrated e-commerce function (e.g. similar to FIGS. 20a-20c). Entries may or may not include expiration alerts or minimum level alerts. There are also controls to initiate the ordering process, and controls to add and remove product from the inventory. A number associated with each entry indicates the quantity of product on hand. For example, in FIG. 22, there are 33 packets of pink latex gloves of a small size located in the office, and a packet of such gloves costs $13.99.

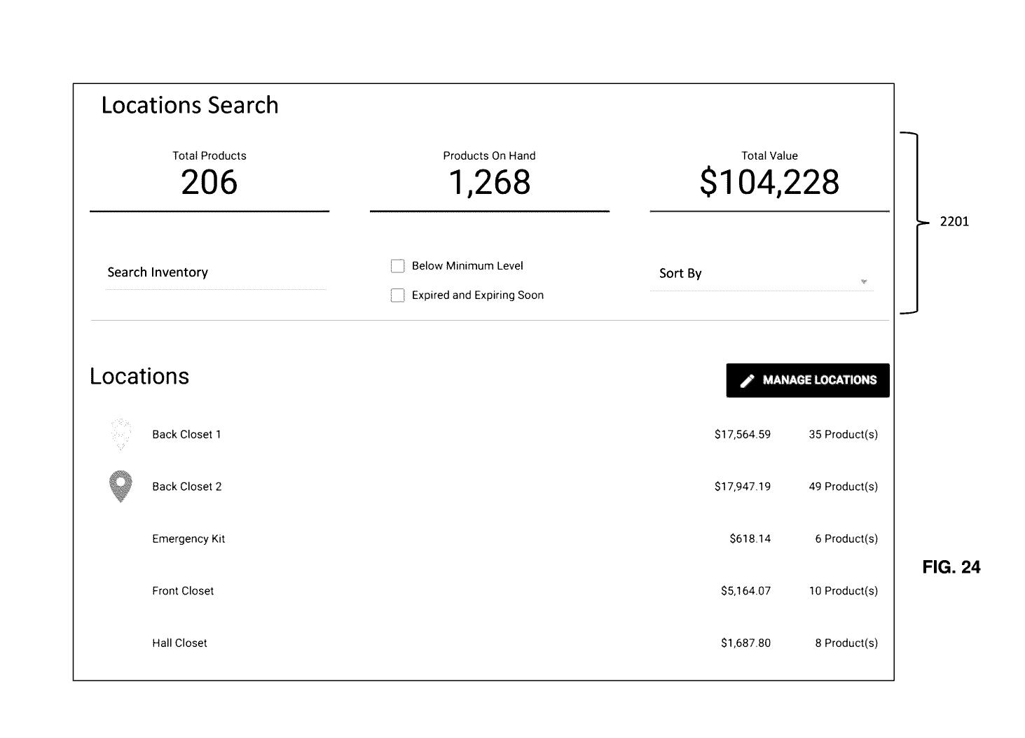

The GUI in FIG. 23 includes the same section 2201. It also shows the products in inventory, but organized by categories. For example, the categories for a dental practice include air cleaning products, anaesthetics, autoclave & sterilization, and burs. Each category entry also displays the total monetary value of the products within the category, and the total number of products within the category.

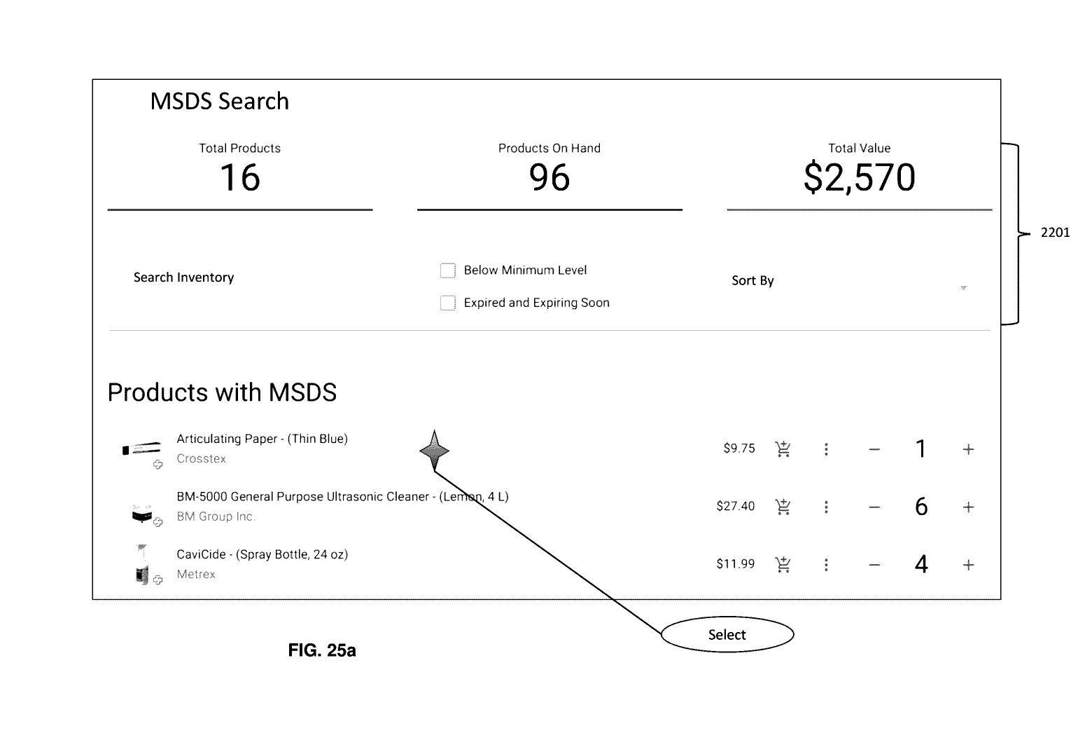

The GUI in FIG. 24 shows the same section 2201. It also shows the products in inventory, but organized by locations. A listing of location entries in the given office are shown (e.g. back closet 1, back closet 2, emergency kit, etc.). Each location entry also displays the total monetary value of the products within the location, and the total number of products within the location.

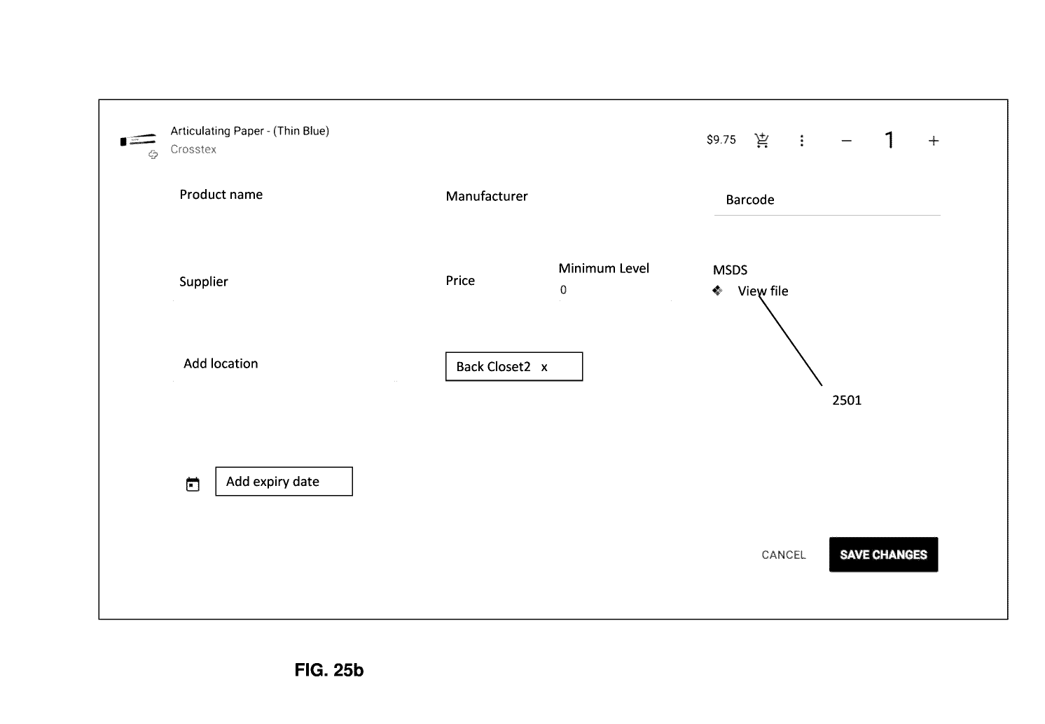

The GUI in FIG. 25a shows the same section 2201. It also shows the products in inventory, but only those products with MSDS information. For example, selecting an entry of the products in FIG. 25a initiates the GUI in FIG. 25b. FIG. 25b shows a GUI of the selected product, which includes a control 2501 for initiating displaying the MSDS of the respective product. In this way the MSDS records for products in a given office are conveniently accessible by users using the IMS.

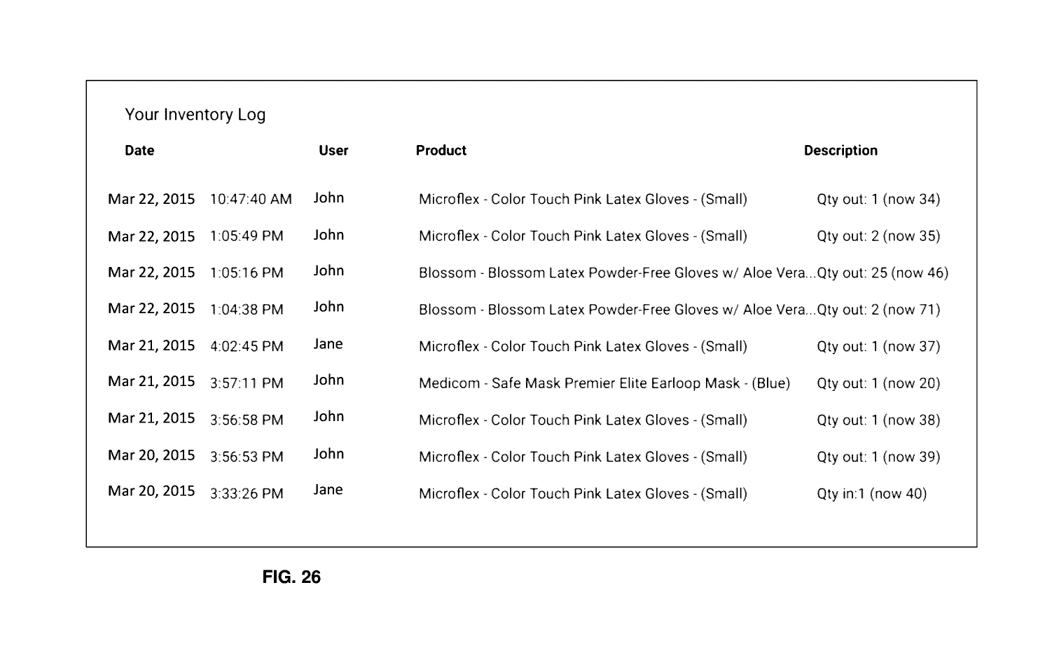

FIG. 26 is another GUI transmitted by the server for display. It shows a log of the inventory actions. For example, each entry includes a date and time stamp, a user responsible for the action, a description of the product, and a description of the action (e.g. the addition or removal of product from inventory, the number added or removed, and the current remaining quantity of such product).

With respect to orders, it will be appreciated that the order status may vary over time. Examples of different statuses include: draft, placed order, backordered, cancelled, and received. The order includes the types or products, the quantity of such products, the unit price, and the total costs. The order also includes an order ID, a shipping address and a billing address. The order also includes a tracking number.

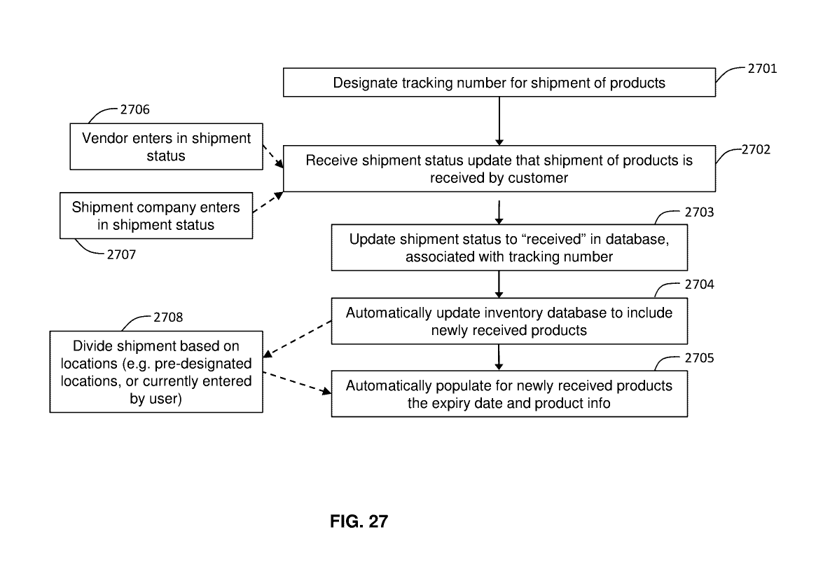

FIG. 27 shows example processor executable instructions performed by the server for using tracking numbers to automatically update inventory database information. At block 2701, the server receives or itself designates a tracking number for a shipment of products for a placed order. At block 2702, the server receives a shipment status update that the shipment is received by the customer who placed the order. For example, the vendor may use their vendor web portal to access the IMS to enter in the shipment status (block 2706), or the shipment company (e.g. the courier or mailing company) enters in the shipment status for the respective tracking number. At block 2703, the server updates the shipment status to “received” in the database, with respect to the tracking number. At block 2704, the server automatically accesses and updates the inventory database to include the newly received products for the given office, and automatically populates the expiry date and other product information for the newly received products, if applicable (block 2705). For example, the other product information may include the location of the product within the office, or amongst different offices. As an example additional step, the shipment of product is divided into at least two locations, and this is reflected in the database. For example, a shipment of 100 latex glove boxes is shipped to a first office, but 30 of the boxes are designated for inventory in a second office in the same health care practice.

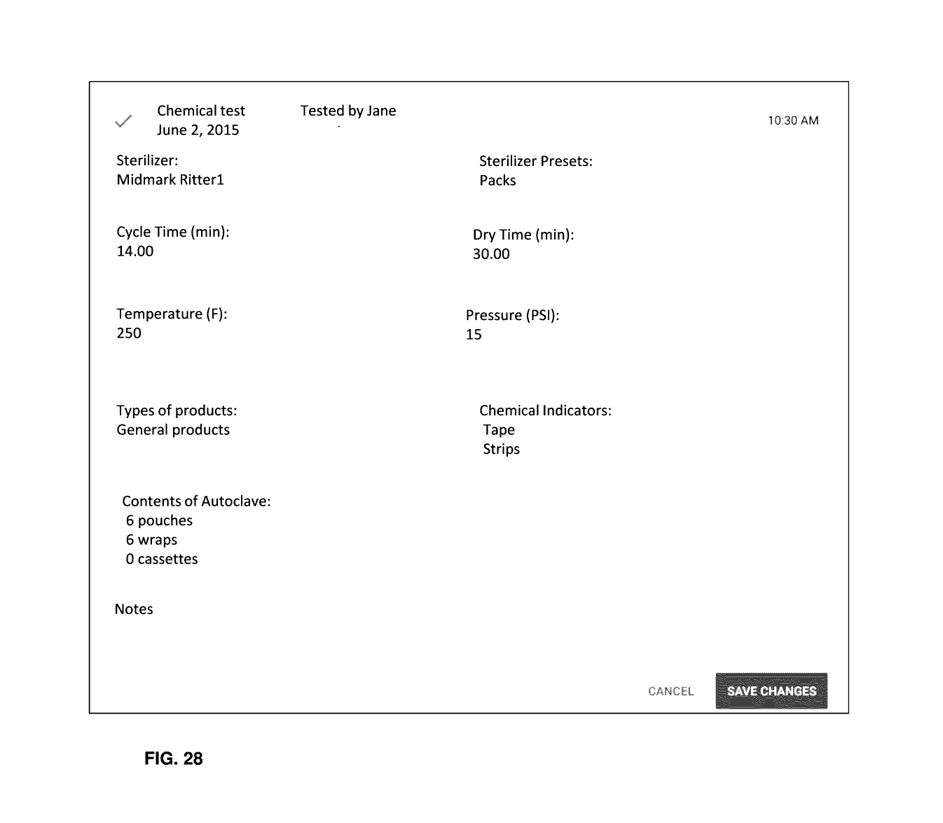

FIG. 28 shows a GUI of details regarding a sterilization test. In this case, the test is a chemical test. It includes information, such as the date of the test, the name of user conducting the test, the time that the test took place, the sterilizer used, the sterilizer presets, the cycle time, the dry time, the temperature, the pressure, the types of products, the chemical indicators used to conduct the test, and the contents of the autoclave. The sterilization module helps a user to review and track past sterilization tests for auditing purposes, as well as to schedule and manage future sterilization tests.

It will be appreciated that autoclaves or sterilization chambers should be tested to ensure they are operating properly. A user can use the controls provided by the server GUI to add in a new entry for a biological test or a new entry for a chemical test. Another GUI, not shown, also is used to show the entire history of sterilization testing, including completed sterilization tests and pending sterilization tests. The GUI in FIG. 28 shows an example of a completed sterilization test entry in a sterilization database (e.g. chemical sterilization database 509).

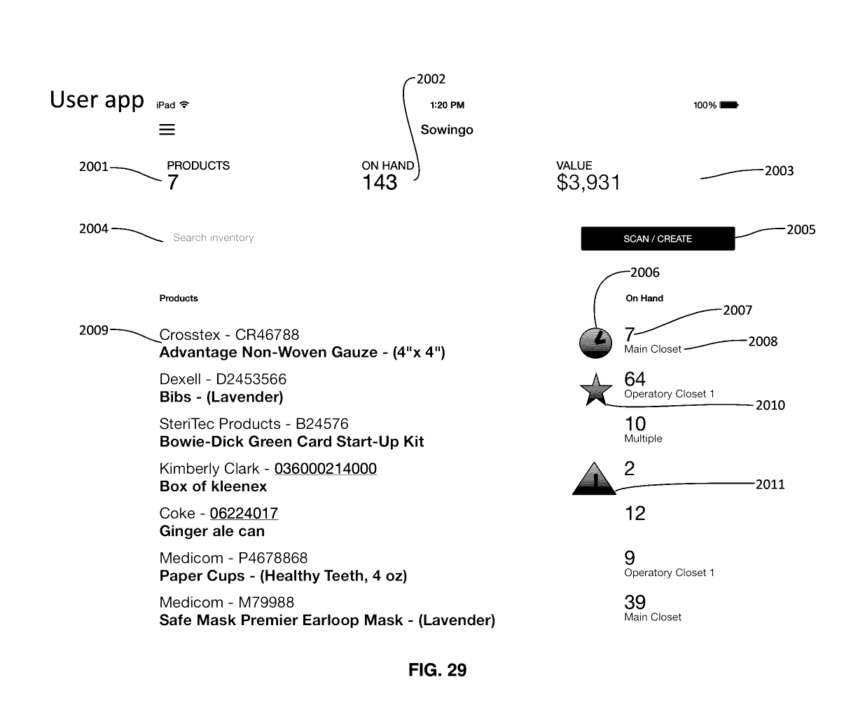

FIGS. 29-36 are example GUIs displayed by the application 113. However, the content of these GUIs may also be provided via an Internet browser.

FIG. 29 shows inventory information including the number of different types of products in inventory 2001, the number of total products in inventory 2002, and the financial or monetary value of the inventory within the office 2003. This is computed by adding the estimated monetary value of each unit of product that is in inventory.

A search bar 2004 is provided to receive a search term and look for items within inventory. A scan/create button 2005 invokes another GUI and process for using a scanning function or adding in a new product, or both.

Continuing with FIG. 29, a listing of products within the inventory are shown. A listing of given products includes the name of the product, the code, the manufacturer, the number of product stored in inventory 2007, and the location of storage within the office 2008. There may also be indicators 2006, 2010, and 2011. The indicator 2006 represents that a product in the inventory is about to expire or has expired. In particular, the indicator 2006 appears when the closest expiry date for the product is approaching. It will be appreciated that different units of the same product may have different expiry dates. The indictor 2010 represents that a product in the inventory is considered a favourite and preferred product. The indicator 2011 indicates that a minimum number of a given product has been reached and more units of the product should be ordered.

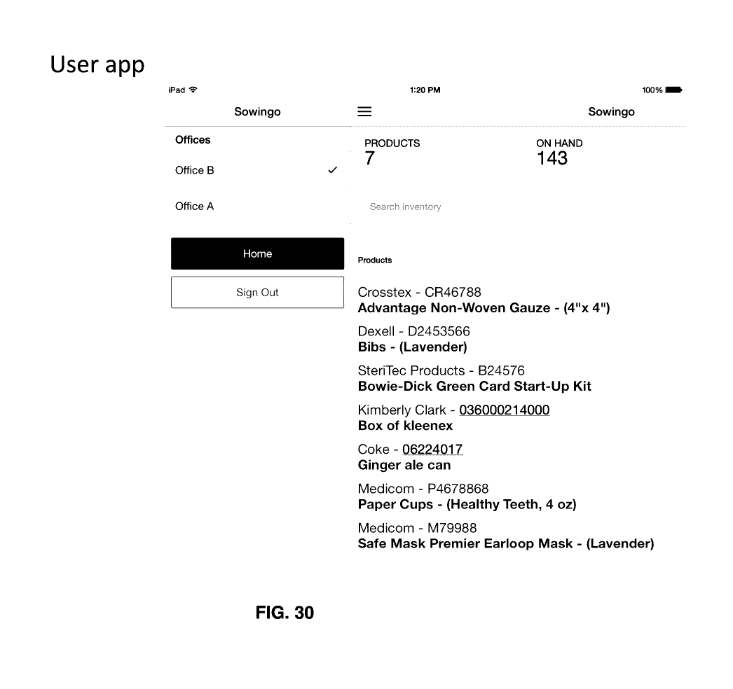

FIG. 30 shows a similar GUI to FIG. 29, but includes controls for switching between offices (if there are more than one office associated with a user).

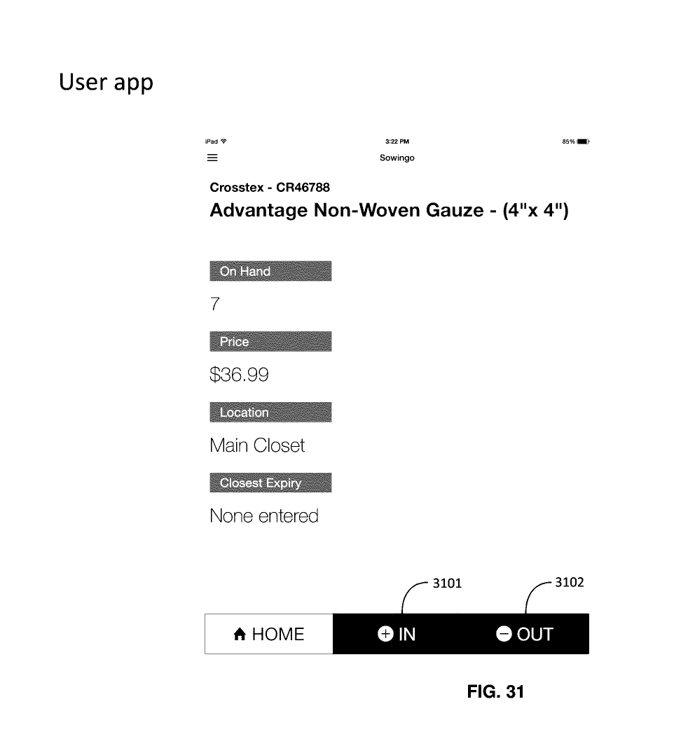

Should an entry or a particular product listing in FIG. 29 be selected, then the product details page is shown, as per FIG. 31. The GUI in FIG. 31 shows information about the product in inventory, and further includes controls 3101, 3102 for respectively adding and removing units from inventory.

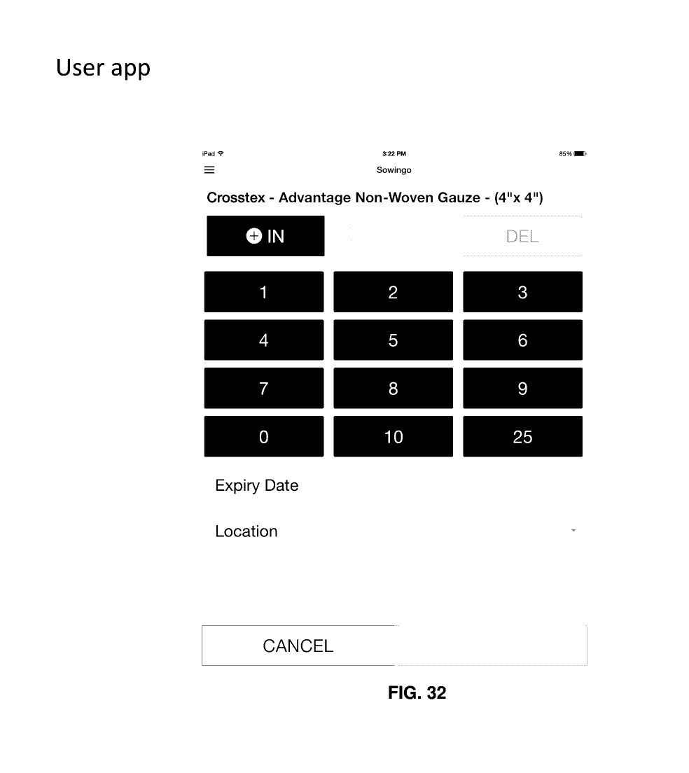

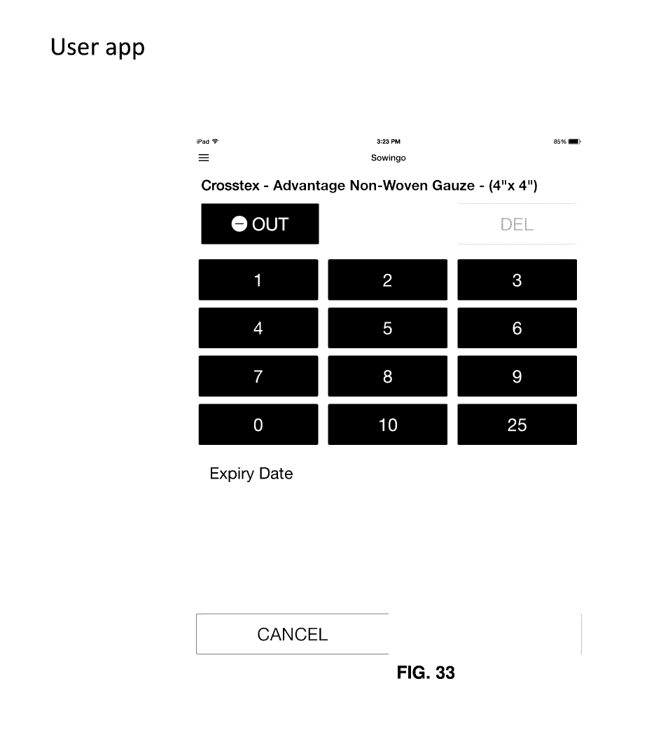

For example, selecting control 3101 invokes the display of the GUI shown in FIG. 32, which includes controls to specify the number of product units that are being added to the inventory. Selecting control 3102 invokes the display of the GUI shown in FIG. 33, which includes controls to specify the number of product units that are being removed from the inventory.

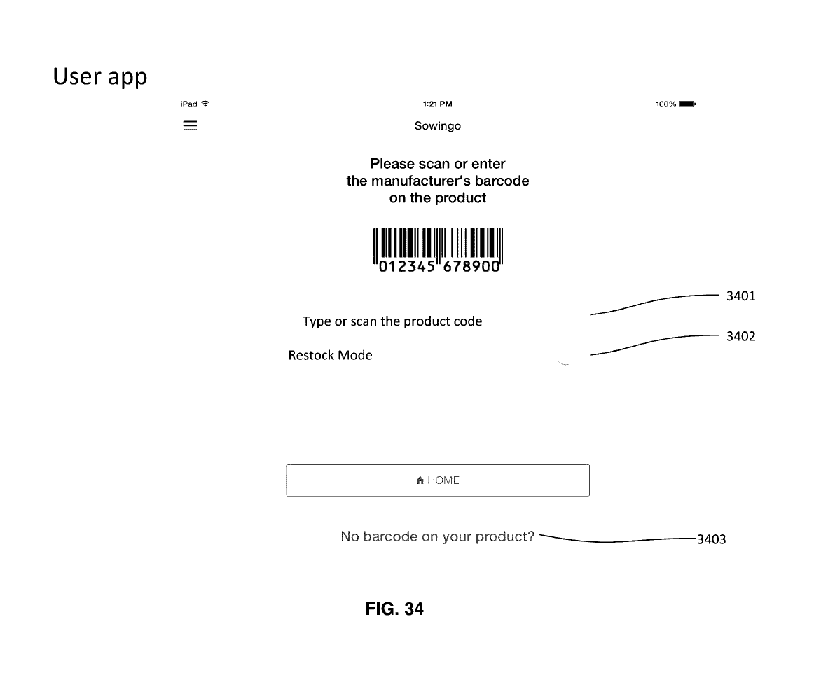

Returning briefly to FIG. 29, selection of the control 2005 invokes the display of the GUI shown in FIG. 34. Hitting the control 2005 also causes the user device, for example, to wirelessly connect to a handheld scanner 116. In another example, hitting the control 2005 causes the user device (e.g. the mobile device 115) to activate its own scanning hardware (e.g. onboard camera device, onboard RFID reader, or onboard barcode reader).

When the GUI of FIG. 34 is displayed, a user can user a handheld scanner 116 or a mobile device 115 to scan a product (e.g. a barcode, an RFID code, or some other product identifier), or may manually enter in data into the field 3401. There is also a control 3402 for the user to switch between a restock mode and a normal mode (i.e. a non-restock mode).

It is appreciated that some products do not have barcodes. Therefore, a user may select the control 3403 to initiate generation of a barcode that can be created and printed. The printed barcodes can then be applied or adhered to products, so that they may be tracked. This is convenient for tacking custom products.

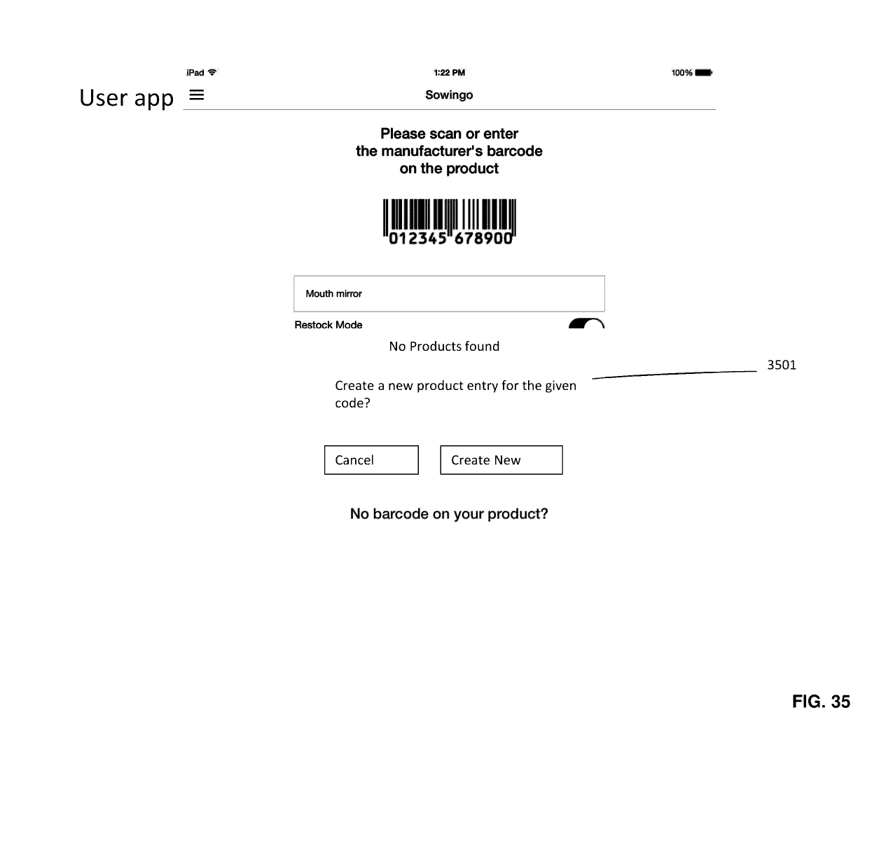

If a product has been inputted into the application, but it does not exist in the products database, then the message 3501 is displayed as per FIG. 35. It prompts the user with the option to create a new product entry.

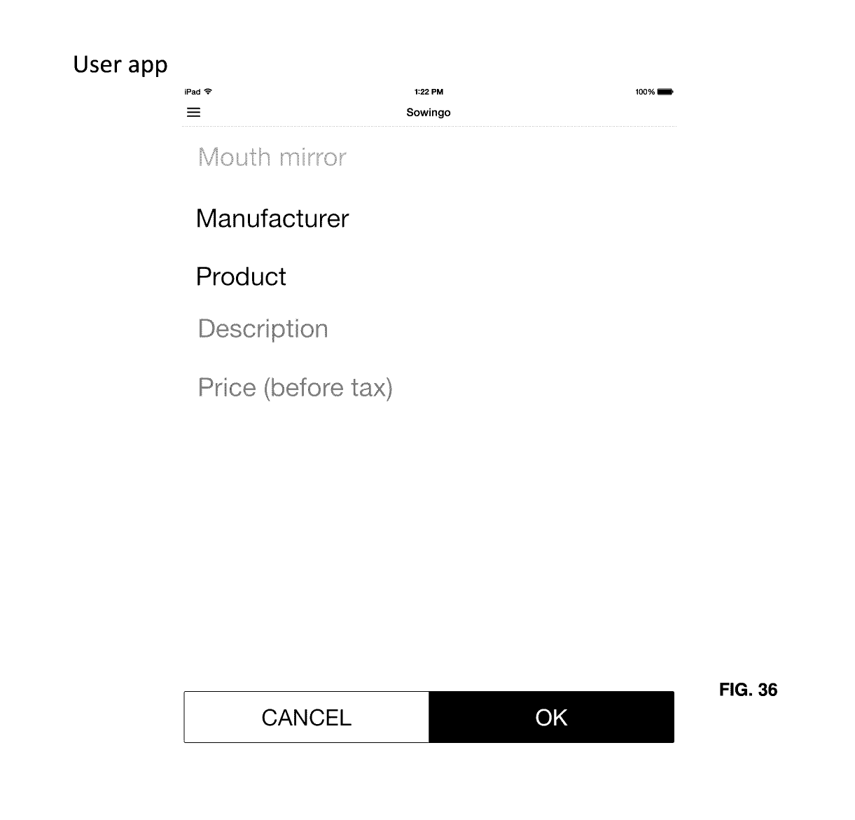

Details of the product entry are inputted through various fields, such as for specifying the manufacturer, the product name, the description, and a price. These data entry fields are shown in the GUI of FIG. 36.

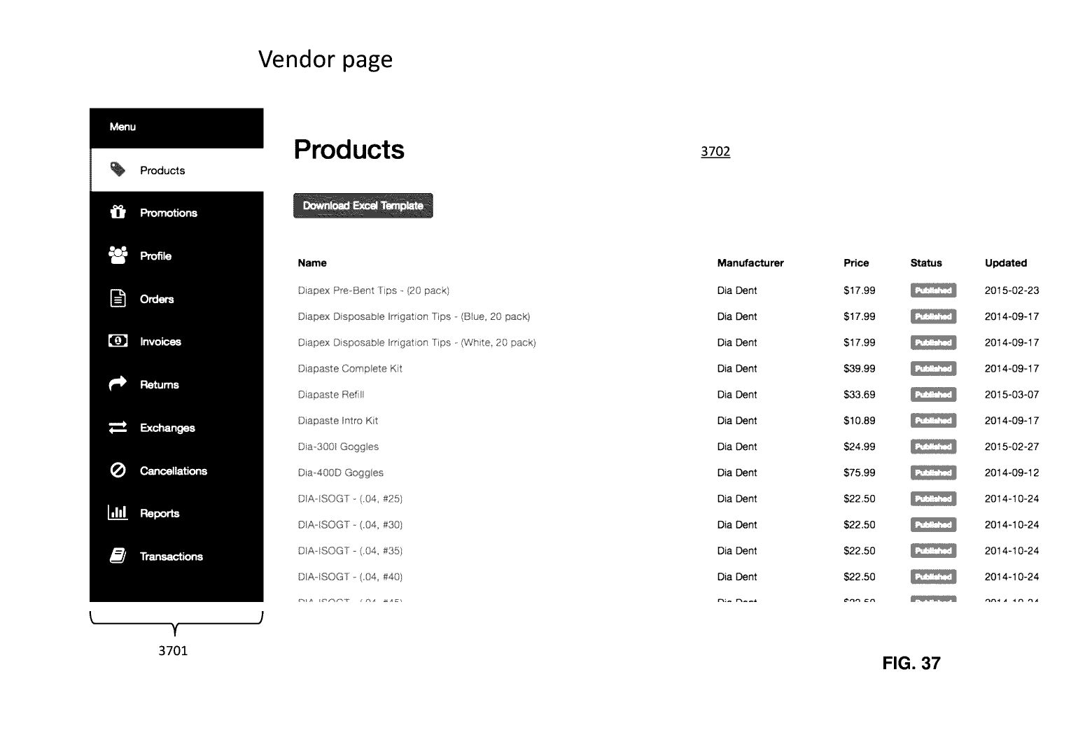

FIGS. 37-41 show GUIs that are accessible to a vendor for managing the sales of products to users.

FIG. 37 is a GUI showing a number of controls 3701 relating to functions and features available to vendors. For example, there is a control for invoking the display of products offered for sale by the vendor, a control for invoking the display of promotions offered by the vendor, a control for invoking the display of managing vendor profile information, a control for invoking the display of placed orders by users to the vendor, a control for invoking the display of invoices for fulfilled orders, a control for invoking the display of returned products, a control for invoking the display of products users wish to exchange with the vendor, a control for invoking the display of order cancellations, a control for invoking the display of reports, and a control for invoking the display of transactions. There may be other controls.

In the example of FIG. 37, the products offered for sale by the vendor is shown 3702. The listing of products includes the name, the manufacturer, the price per unit, the status (e.g. published or not on the e-commerce marketplace), and the date information related to the product was last updated.

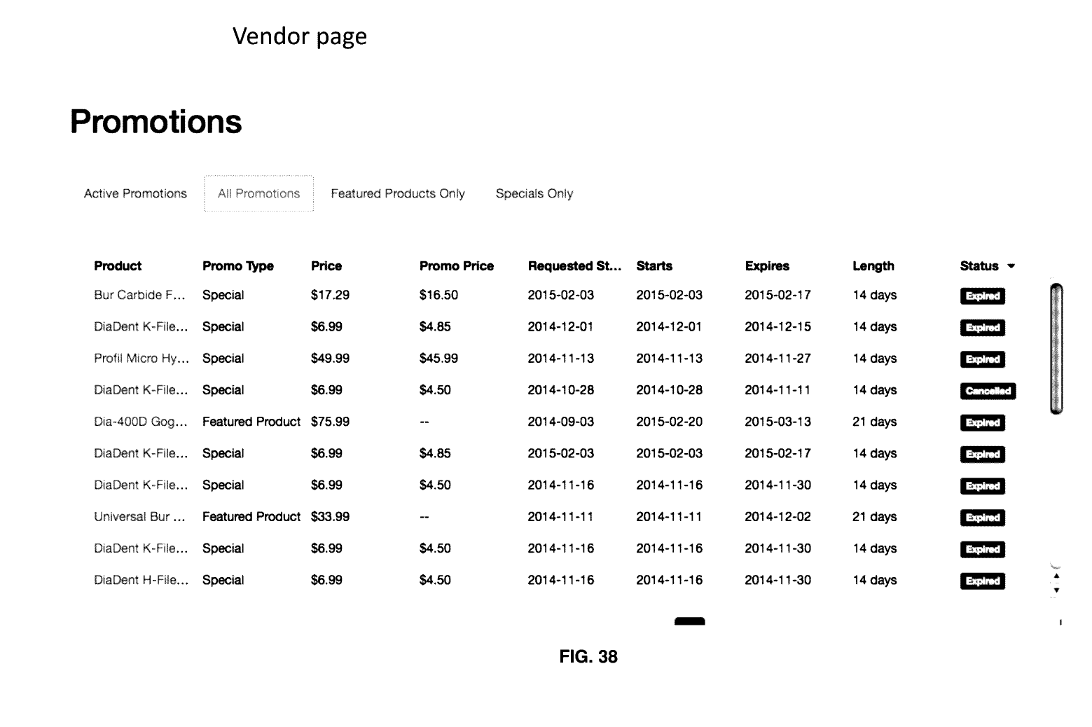

After the promotions control is selected, the GUI of FIG. 38 is displayed. The GUI in FIG. 38 shows the different products that were promoted or are promoted, including information such as: the promotion type, the promotion price, the start date of the promotion, the end date of the promotion, the length of the promotion (e.g. in days), and a status indicator (e.g. expired, cancelled, active, etc.).

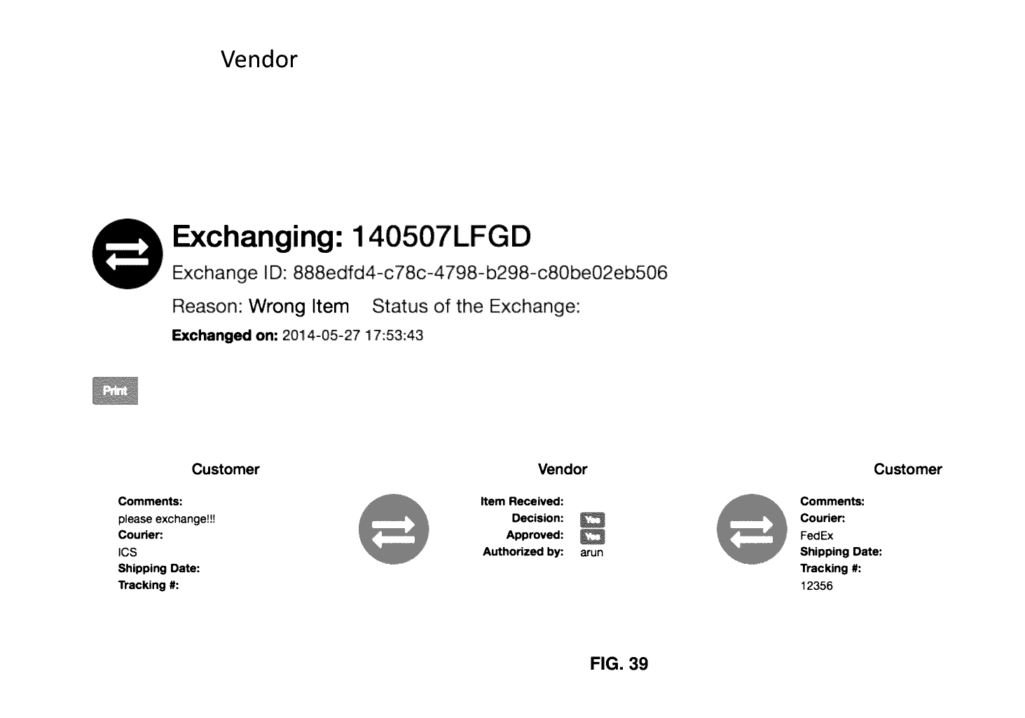

FIG. 39 is a GUI that shows information about a specific exchange request from a customer. It includes information about the product being exchanged, the reason for the exchange, the status of the exchange, the courier information associated with the exchanged and other information.

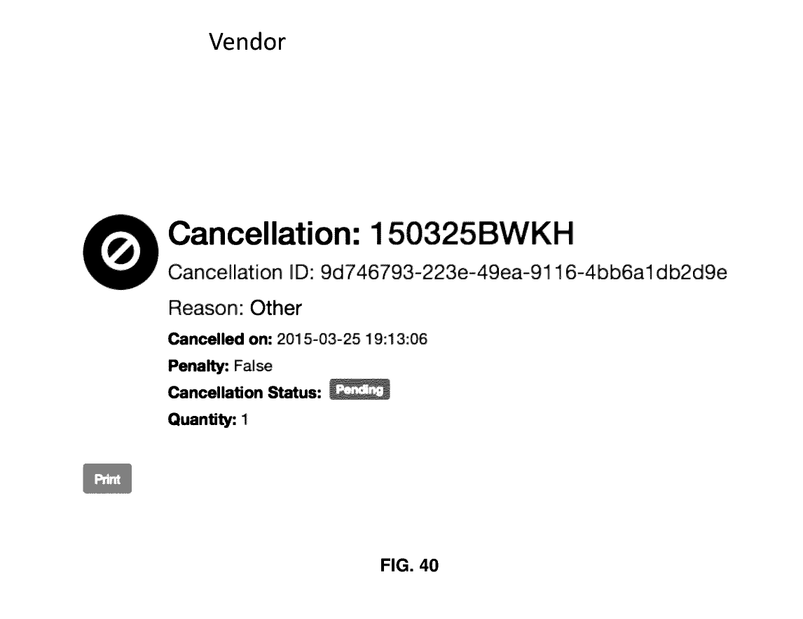

FIG. 40 is a GUI that shows information about a specific cancellation request. The information includes the reason for the cancellation, information about the order, whether or not there is a penalty, and a status of the cancellation.

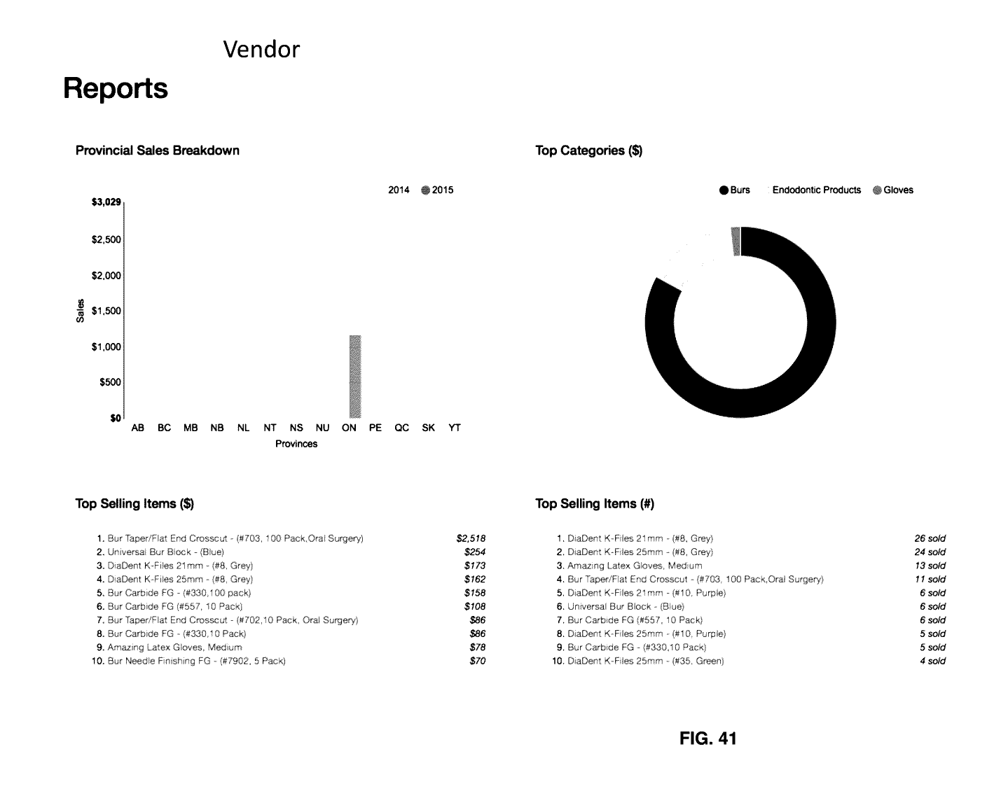

FIG. 41 is a GUI that shows reports for the vendor. It includes, for example, the breakdown of sales by location (e.g. state, province, etc.), the top product categories of sales, the top selling items by revenue, and the top selling products based on quantity.

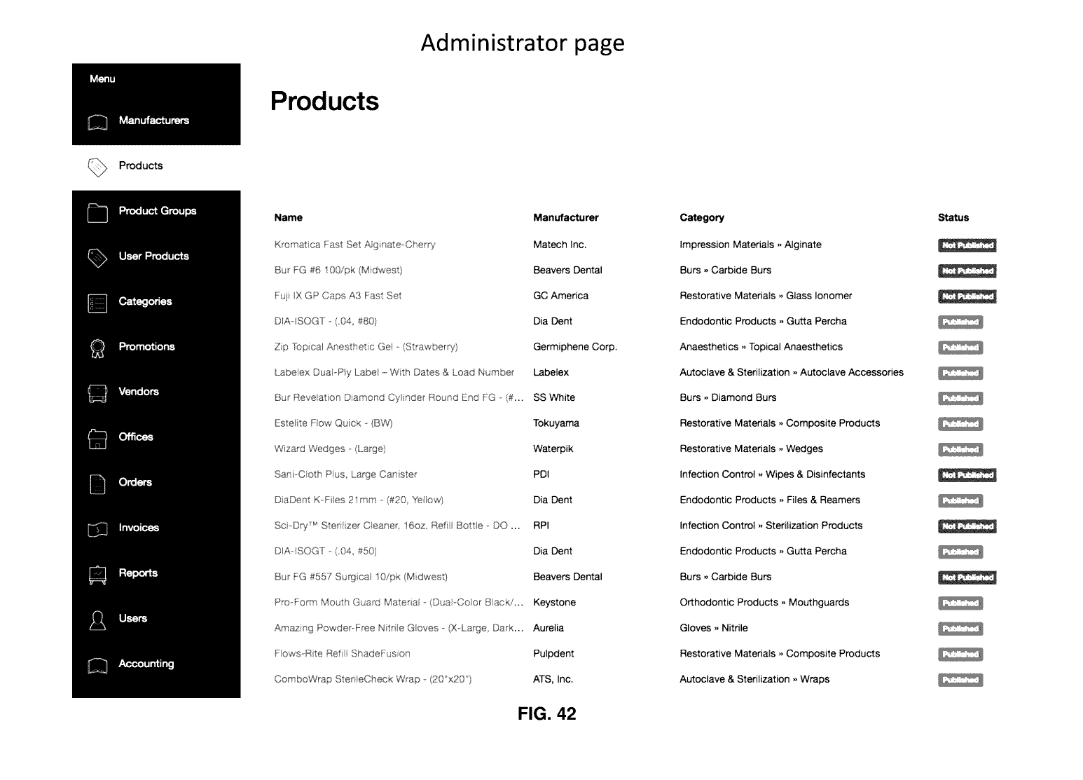

FIG. 42 is a GUI that is able to access information that is accessible to vendor or the user, or both. Therefore, many of the controls and information shown to the administrator is similar or the same as those described above.

Example embodiments and example aspects of the above systems and methods are provided below.

In an example embodiment, a computing system is configured to manage inventory for a health care office. The computing system comprises: a processor; a communication device; and a memory configured to store a procedure profile database, each procedure profile in the procedure profile database including product usage information of products expected to be consumed in a given procedure. The processor is configured to execute instructions to at least: obtain a digital schedule of future health care procedures, the digital schedule identifying a date for each procedure; compute an amount of a product that will be consumed by comparing each procedure against a corresponding procedure profile obtained from the procedure profile database, the product usage information of at least one or more of procedure profiles including a product identifier of the product and a corresponding quantity of the product; compute how many days later from a current date that a number of currently available units of the product in an inventory of the health care office will run out based on the computed amount of the product that will be consumed in the future; determine a date of when to order replacement units of the product and a quantity of replacement units of the product based on at least the predicted number of days later from the current date that the currently available units of the product in the inventory will run out; and provide a graphical user interface to order the replacement units by the determined date and for the determined quantity.

In another example embodiment, a server system for managing inventory comprises: memory storing databases, including a product database, an order database, and an inventory database; a communication device able to communicate over Internet with one or more user devices and one or more vendor computing device; and one or more processors. The one or more processors are configured to execute instructions to at least: generate and transmit a graphical user interface (GUI) via the communication device that is displayable on a given user device, the GUI comprising a product listed in the product database; receive via the communication device a command using the GUI to order the product; generate an order entry in the order database for the product, the order entry including an ordered quantity of the product, a tracking number and an order status, the order entry accessible to a given vendor computing device; receive via the communication device a message able to be transmitted by the given vendor computing device, the message comprising a shipped status associated with the tracking number; access the order database to update the order entry to have a received status; and access and automatically update the inventory database to have an updated quantity of the product in inventory that includes the ordered quantity of the product.

In another example aspect, the one or more processors are configured to further update and transmit the GUI showing the updated quantity of product in inventory.

In another example aspect, the product listed in the GUI is displayed alongside an alert generated by the server system.

In another example aspect, the inventory database includes a product entry of the product, an associated minimum quantity, and a current quantity of the product in inventory; the current quantity is equal to or less than the associated minimum quantity; and in response, the server system generates a minimum level alert associated with the product for display in the GUI.

In another example aspect, the memory further comprises a scheduling database and a procedure profile database; and the one or more processors further configured to: obtain a schedule of future health care procedures by accessing the scheduling database, the schedule identifying a date for each procedure; compute an amount of a product that will be consumed by comparing each procedure against a corresponding procedure profile obtained from the procedure profile database, the product usage information of at least one or more of procedure profiles including a product identifier of the product and a corresponding usage quantity of the product; compute how many days later from a current date that a number of currently available units of the product in an inventory of the health care office will run out based on the computed amount of the product that will be consumed in the future; determine a date of when to order replacement units of the product and a quantity of replacement units of the product based on at least the predicted number of days later from the current date that the currently available units of the product in the inventory will run out; and generate a prediction alert to order the replacement units by the determined date and for the determined quantity, the prediction alert for display in the GUI.

In another example aspect, the inventory database includes a product entry of the product and an associated expiry date of certain units of the product; the associated expiry date is within one or more days from a current date, or the associated expiry date has passed; and in response, the server system generates an expiry date alert associated with the product for display in the GUI.

In another example aspect, responsive to receiving scanned barcode data via the communication device, the server system identifies the product associated with the scanned barcode data by accessing the inventory database and displays the GUI showing the product.

In another example aspect, responsive to receiving scanned radio frequency identification (RFID) data via the communication device, the server system identifies the product associated with the scanned RFID data by accessing the inventory database and displays the GUI showing the product.

In another example aspect, responsive to receiving a digital image via the communication device, the server system identifies the product associated with the digital image by accessing the inventory database and displays the GUI showing the product.

In another example aspect, the server system further comprises an accounting database stored in the memory, and after automatically updating the inventory database to have the updated quantity of the product in inventory, the server system automatically updates the accounting database to have an updated monetary value associated with total current inventory.

In another example aspect, the server system further generates and transmits another GUI that includes the updated total monetary value.

In another general example embodiment, a method performed by at least a user device is provided. The method includes: a communication system on the user device receiving data regarding inventory that is stored in an inventory database on a server; displaying a graphical user interface (GUI) comprising the data on a display screen of the user device; receiving, via the GUI, an input to activate a scanning device; the scanning device scanning a tag or an object to obtained scanned data; transmitting the scanned data via the communication system and, in response, receiving product data corresponding to the scanned data; and receiving, via the GUI, an input to modify an entry in the inventory database in relation to the product data.

In an example aspect, the user device comprises the scanning device.

In another example aspect, the user device comprises a wireless radio for data communication with the scanning device, and the scanning device is a separate handheld scanner. For example the wireless radio is a BlueTooth radio.

In another example aspect, the received product data comprises an indication that the scanned data does not correspond to an existing product on the server, and after receiving the input to modify the entry in the inventory database, the display screen displays the GUI showing that a new product corresponding to the scanned data is added to the inventory database.

The steps or operations in the flow charts described herein are just for example. There may be many variations to these steps or operations without departing from the principles described herein. For instance, the steps may be performed in a differing order, or steps may be added, deleted, or modified.

The GUIs and graphical controls and components shown and described herein are just for example. There may be many variations to these GUIs without departing from the principles described herein. For instance, the graphical elements may be positioned in differing positions, may have different shapes, or graphical elements may be added, deleted, or modified.

It will be appreciated that the features of the systems and methods for managing inventory for health care practices and organizations are described herein with respect to example embodiments. However, these features may be combined with different features and different embodiments of these systems and methods, although these combinations are not explicitly stated.

While the basic principles of these inventions have been described and illustrated herein it will be appreciated by those skilled in the art that variations in the disclosed arrangements, both as to their features and details and the organization of such features and details, may be made without departing from the spirit and scope thereof. Accordingly, the embodiments described and illustrated should be considered only as illustrative of the principles of the inventions, and not construed in a limiting sense.

1. A method performed by a computing system to manage inventory for a health care office, comprising:
obtaining a digital schedule of future health care procedures, the digital schedule identifying a date for each procedure;
computing an amount of a product that will be consumed by comparing each procedure against a corresponding procedure profile obtained from a procedure profile database, each procedure profile including product usage information of products expected to be consumed in a given procedure, the product usage information including a product identifier of the product and a corresponding quantity of the product;
computing how many days later from a current date that a number of currently available units of the product in an inventory of the health care office will run out based on the computed amount of the product that will be consumed in the future;
determining a date of when to order replacement units of the product and a quantity of replacement units of the product based on at least the predicted number of days later from the current date that the currently available units of the product in the inventory will run out; and
providing a graphical user interface to order the replacement units by the determined date and for the determined quantity.

2. A computing system configured to manage inventory for a health care office, comprising:
a processor;
a communication device;
a memory configured to store a procedure profile database, each procedure profile in the procedure profile database including product usage information of products expected to be consumed in a given procedure; and
wherein the processor is configured to execute instructions to at least:
obtain a digital schedule of future health care procedures, the digital schedule identifying a date for each procedure;
compute an amount of a product that will be consumed by comparing each procedure against a corresponding procedure profile obtained from the procedure profile database, the product usage information of at least one or more of procedure profiles including a product identifier of the product and a corresponding quantity of the product;
compute how many days later from a current date that a number of currently available units of the product in an inventory of the health care office will run out based on the computed amount of the product that will be consumed in the future;
determine a date of when to order replacement units of the product and a quantity of replacement units of the product based on at least the predicted number of days later from the current date that the currently available units of the product in the inventory will run out; and
provide a graphical user interface to order the replacement units by the determined date and for the determined quantity.

3. A server system for managing inventory comprising:
memory storing databases, including a product database, an order database, and an inventory database;
a communication device able to communicate over Internet with one or more user devices and one or more vendor computing device; and
one or more processors configured to execute instructions to at least:
generate and transmit a graphical user interface (GUI) via the communication device that is displayable on a given user device, the GUI comprising a product listed in the product database;
receive via the communication device a command using the GUI to order the product;
generate an order entry in the order database for the product, the order entry including an ordered quantity of the product, a tracking number and an order status, the order entry accessible to a given vendor computing device;
receive via the communication device a message able to be transmitted by the given vendor computing device, the message comprising a shipped status associated with the tracking number;
access the order database to update the order entry to have a received status; and
access and automatically update the inventory database to have an updated quantity of the product in inventory that includes the ordered quantity of the product.

4. The server system of claim 3 wherein the one or more processors are configured to further update and transmit the GUI showing the updated quantity of product in inventory.

5. The server system of claim 3 wherein the product listed in the GUI is displayed alongside an alert generated by the server system.

6. The server system of claim 3 wherein: the inventory database includes a product entry of the product, an associated minimum quantity, and a current quantity of the product in inventory; the current quantity is equal to or less than the associated minimum quantity; and in response, the server system generates a minimum level alert associated with the product for display in the GUI.

7. The server system of claim 3 wherein the memory further comprises a scheduling database and a procedure profile database; and the one or more processors further configured to:
obtain a schedule of future health care procedures by accessing the scheduling database, the schedule identifying a date for each procedure;
compute an amount of a product that will be consumed by comparing each procedure against a corresponding procedure profile obtained from the procedure profile database, the product usage information of at least one or more of procedure profiles including a product identifier of the product and a corresponding usage quantity of the product;
compute how many days later from a current date that a number of currently available units of the product in an inventory of the health care office will run out based on the computed amount of the product that will be consumed in the future;
determine a date of when to order replacement units of the product and a quantity of replacement units of the product based on at least the predicted number of days later from the current date that the currently available units of the product in the inventory will run out; and
generate a prediction alert to order the replacement units by the determined date and for the determined quantity, the prediction alert for display in the GUI.

8. The server system of claim 3 wherein the inventory database includes a product entry of the product and an associated expiry date of certain units of the product; the associated expiry date is within one or more days from a current date, or the associated expiry date has passed; and in response, the server system generates an expiry date alert associated with the product for display in the GUI.

9. The server system of claim 3 wherein responsive to receiving scanned barcode data via the communication device, the server system identifies the product associated with the scanned barcode data by accessing the inventory database and displays the GUI showing the product.

10. The server system of claim 3 wherein responsive to receiving scanned radio frequency identification (RFID) data via the communication device, the server system identifies the product associated with the scanned RFID data by accessing the inventory database and displays the GUI showing the product.

11. The server system of claim 3 wherein responsive to receiving a digital image via the communication device, the server system identifies the product associated with the digital image by accessing the inventory database and displays the GUI showing the product.

12. The server system of claim 3 further comprising an accounting database stored in the memory, and after automatically updating the inventory database to have the updated quantity of the product in inventory, the server system automatically updating the accounting database to have an updated monetary value associated with total current inventory.

13. The server system of claim 12 further generating and transmitting another GUI that includes the updated total monetary value.

14. A method performed by at least a user device comprising:
a communication system on the user device receiving data regarding inventory that is stored in an inventory database on a server;
displaying a graphical user interface (GUI) comprising the data on a display screen of the user device;
receiving, via the GUI, an input to activate a scanning device;
the scanning device scanning a tag or an object to obtained scanned data;
transmitting the scanned data via the communication system and, in response, receiving product data corresponding to the scanned data; and
receiving, via the GUI, an input to modify an entry in the inventory database in relation to the product data.

15. The method of claim 14 wherein the user device comprises the scanning device.

16. The method of claim 14 wherein user device comprises a wireless radio for data communication with the scanning device, and the scanning device is a separate handheld scanner.

17. The method of claim 14 wherein the received product data comprises an indication that the scanned data does not correspond to an existing product on the server, and after receiving the input to modify the entry in the inventory database, the display screen displays the GUI showing that a new product corresponding to the scanned data is added to the inventory database.
